(19) United States
(12) Patent Application Publication
(10) Pub. No.: US 2023/0061534 A1
(43) Pub. Date: Mar. 2, 2023

(54) MINIMALLY INVASIVE HISTOTRIPSY SYSTEMS AND METHODS

(71) Applicant: HISTOSONICS, INC., Plymouth, MN (US)

(72) Inventor: Joshua STOPEK, Ann Arbor, MI (US)

(21) Appl. No.: 17/904,326

(22) PCT Filed: Mar. 8, 2021
(86) PCT No.: PCT/US2021/021368

§ 371 Date: Aug. 16, 2022

Related U.S. Application Data
(60) Provisional application No. 62/986,410, filed on Mar. 6, 2020.

(51) Int. Cl. A61B 34/20; A61B 34/00

(52) U.S. Cl. CPC A61B 34/20; A61B 34/25; A61B 2017/00477

(57) ABSTRACT

A histotripsy therapy system configured for the treatment of tissue is provided, which may include any number of features. Provided herein are systems and methods that provide efficacious non-invasive and minimally invasive therapeutic, diagnostic and research procedures. In particular, provided herein are optimized systems and methods that provide targeted, efficacious histotripsy in a variety of different regions and under a variety of different conditions without causing undesired tissue damage to intervening/non-target tissues or structures.

### CROSS REFERENCE TO RELATED APPLICATIONS

This application claims the benefit under 35 U.S.C. § 119 of U.S. Provisional Patent Appin. No. 62/986,410, filed Mar. 6, 2020, titled “Minimally Invasive Histotripsy Systems and Methods”, the disclosure of which is incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present disclosure details novel high intensity therapeutic ultrasound (HITU) systems configured to produce acoustic cavitation, methods, devices and procedures for the minimally and non-invasive treatment of healthy, diseased and/or injured tissue. The acoustic cavitation systems and methods described herein, also referred to Histotripsy, may include transducers, drive electronics, positioning robotics, imaging systems, and integrated treatment planning and control software to provide comprehensive treatment and therapy for soft tissues in a patient.

### BACKGROUND

Histotripsy, or pulsed ultrasound cavitation therapy, is a technology where extremely short, intense bursts of acoustic energy induce controlled cavitation (microbubble formation) within the focal volume. The vigorous expansion and collapse of these microbubbles mechanically homogenizes cells and tissue structures within the focal volume. This is a very different end result than the coagulative necrosis characteristic of thermal ablation. To operate within a non-thermal, Histotripsy realm; it is necessary to deliver acoustic energy in the form of high amplitude acoustic pulses with low duty cycle.

Compared with conventional focused ultrasound technologies, Histotripsy has important advantages: 1) the destructive process at the focus is mechanical, not thermal; 2) cavitation appears bright on ultrasound imaging thereby confirming correct targeting and localization of treatment; 3) treated tissue generally, but not always, appears darker (more hypoechoic) on ultrasound imaging, so that the operator knows what has been treated; and 4)

Histotripsy produces lesions in a controlled and precise manner. It is important to emphasize that unlike thermal ablative technologies such as microwave, radiofrequency, high-intensity focused ultrasound (HIFU) cryo or radiation, Histotripsy relies on the mechanical action of cavitation for tissue destruction and not on heat, cold or ionizing energy.

### SUMMARY

A method of treating tissue of a patient with a robotic surgical system is provided, comprising the steps of identifying a target tissue location with an imaging sub-system of the robotic surgical system, preparing the target tissue location for histotripsy therapy with a laparascopic sub-system of the robotic surgical system, and delivering histotripsy therapy to the prepared target tissue location with a histotripsy sub-system of the robotic surgical system.

In some embodiments, the imaging sub-system comprises an endoscopic robotic system, an ultrasound imaging system, a CT imaging system, a cone-CT imaging system, an augmented or enriched multi-modality imaging system, and/or a fluoroscopy imaging system.

In some embodiments, the imaging sub-system comprises an imaging device disposed on a robotic arm of the robotic surgical system.

In one implementation, preparing the target tissue location further comprises resecting intervening tissues between an exterior of the patient and the target tissue location.

In another embodiment, the target tissue location comprises a hollow/lumenal body organ, vessel, or lumen, and wherein preparing the target tissue location further comprises fluidizing the target tissue location with the laparascopic sub-system to create an acoustic window within the target tissue location and/or pathway to the location.

In some embodiments, delivering histotripsy therapy further comprises lysing or liquefying the target tissue location.

In one implementation, the target tissue location comprises a first tissue structure and a second tissue structure, wherein delivering histotripsy therapy further comprises lysing or liquefying the first tissue structure but not the second tissue structure.

In one embodiment, the first tissue structure comprises soft tissue, cancerous tissue, tumor tissue, blood vessels, or ducts including bile ducts.

In one embodiment, delivering histotripsy further comprises evaluating a cavitation threshold at one or more locations within the target tissue location, and optimizing histotripsy therapy parameters based on the evaluated cavitation threshold.

In some embodiments, the histotripsy sub-system is disposed on a robotic arm comprising 3 or more degrees of freedom.

In one example, the robotic surgical system comprises a cart/column based surgical system.

In another embodiment, the robotic surgical system comprises a bed-based surgical system.

A surgical system is provided, comprising at least one imaging sub-system configured to identify a target tissue location of a patient, a laparascopic sub-system disposed on at least one robotic arm of the surgical system, the laparascopic sub-system being configured to prepare the target tissue location for histotripsy therapy, and a histotripsy sub-system disposed on at least one robotic arm of the surgical system, the histotripsy sub-system being configured to deliver histotripsy therapy to the prepared target tissue location.

In some embodiments, the imaging sub-system comprises an endoscopic robotic system, an ultrasound imaging system, a CT imaging system, a cone-CT imaging system, an augmented or enriched multi-modality imaging system, and/or a fluoroscopy imaging system.

A method of treating tissue of a patient with a robotic surgical system is provided, comprising the steps of identifying a target tissue location with an imaging sub-system of the robotic surgical system; preparing the target tissue location for surgery with a histotripsy sub-system of the robotic surgical system; and performing a surgical operation on the prepared target tissue location with a laparoscopic sub-system of the robotic surgical system.

In some embodiments, the imaging sub-system comprises an endoscopic robotic system, an ultrasound imaging system, a CT imaging system, a cone-CT imaging system, an augmented or enriched multi-modality imaging system, and/or a fluoroscopy imaging system.

In some embodiments, the imaging sub-system comprises an imaging device disposed on a robotic arm of the robotic surgical system.

In one implementation, preparing the target tissue location further comprises skeletonizing soft tissue within the target tissue location with the histotripsy sub-system.

In some implementations, preparing the target tissue location for surgery with the histotripsy sub-system further comprises evaluating a cavitation threshold at one or more locations within the target tissue location, and optimizing histotripsy therapy parameters based on the evaluated cavitation threshold; and delivering histotripsy therapy to lyse or liquefy only a first tissue structure of the target tissue location and not a second tissue structure of the target tissue location.

In one embodiment, the first tissue structure comprises soft tissue, cancerous tissue, tumor tissue, blood vessels, or ducts including bile ducts

In some embodiments, the histotripsy sub-system is disposed on a robotic arm comprising 3 or more degrees of freedom.

In some embodiments, the robotic surgical system comprises a cart/column based surgical system.

In other embodiments, the robotic surgical system comprises a bed-based surgical system.

In some examples, performing the surgical operation further comprises resecting one or more tissues of the target tissue location with the laparascopic sub-system. In one embodiment, resecting further comprises performing energy-based cutting, sealing and/or using ligation devices, using monopolar or bipolar devices, performing endostapling and/or endoclipping.

In one example the target tissue location comprises a liver, a kidney, a pancreas, a head/neck, a thyroid, a spleen, a prostate, a heart, lungs, a central or peripheral vasculature, a spinal cord, and/or brain tissue.

In some embodiments, the surgery further comprises dividing one or more lobes or segments of the liver.

In one embodiment, the divided lobes or segments of the liver are removed from the body.

A surgical system is provided, comprising at least one imaging sub-system configured to identify a target tissue location of a patient; a histotripsy sub-system disposed on at least one robotic arm of the surgical system, the histotripsy sub-system being configured to prepare the target tissue location for surgery, a laparascopic sub-system disposed on at least one robotic arm of the surgical system, the laparascopic sub-system being configured to performing a surgical operation on the prepared target tissue location.

In some embodiments, the imaging sub-system comprises an endoscopic robotic system.

In one embodiment, the imaging sub-system comprises an ultrasound imaging system.

In another embodiment, the imaging sub-system comprises a CT imaging system.

In some embodiments, the imaging sub-system comprises an augmented or enriched multi-modality imaging system.

In other embodiments, the imaging sub-system comprises a fluoroscopy imaging system.

A method of treating tissue with a robotic surgical system is provided, comprising the steps of accessing a target hollow organ location with an endoscopic robotic system of the robotic surgical system, fluidizing the target hollow organ location to create an acoustic window within the target hollow organ location, and delivering histotripsy therapy to the fluidized target hollow organ location with a histotripsy sub-system of the robotic surgical system.

In some embodiments, the target hollow organ comprises a lung or a colon.

In some embodiments, fluidizing the target hollow organ location comprises fluidizing the target hollow organ location with the endoscopic robotic system.

In one embodiment, the method further comprises performing the accessing, fluidizing, and delivering steps under real-time imaging guidance.

In one example, the real-time imaging guidance comprises CT, fluoro and/or cone beam CT data/imaging.

In one embodiment, the real-time imaging guidance comprises ultrasound imaging.

A method of treating tissue with a robotic surgical system is provided, comprising the steps of accessing a target hollow organ location with a laparascopic robotic system of the robotic surgical system, fluidizing a body cavity adjacent to the target hollow organ location to create an acoustic window to the target hollow organ location, and delivering histotripsy therapy to the target hollow organ location with a histotripsy sub-system of the robotic surgical system.

In some embodiments, the target hollow organ comprises a lung or a colon.

In one embodiment, fluidizing the body cavity comprises fluidizing the body cavity with the laparascopic robotic system.

In another embodiment, the method further comprises performing the accessing, fluidizing, and delivering steps under real-time imaging guidance.

In some embodiments, the imaging sub-system comprises an endoscopic robotic system, an ultrasound imaging system, a CT imaging system, a cone-CT imaging system, an augmented or enriched multi-modality imaging system, and/or a fluoroscopy imaging system.

In one embodiment, the method further comprises fluidizing the target hollow organ location to create an acoustic window within the target hollow organ location; and delivering histotripsy therapy within the fluidized target hollow organ location with the histotripsy sub-system.

In some examples, the target organ location is visualized in real-time using one or more modalities including ultrasound, X-ray based imaging and/or optical imaging.

In one embodiment, a position of a histotripsy focus may be updated based on feedback provided by real-time imaging guidance.

In another embodiment, an endoscopic/laparascopic robot allows manipulation of the position of real-time imaging guidance, one or more surgical instruments/tools and the histotripsy therapy transducer at the same time, using two or more robotic arms.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1B illustrate an ultrasound imaging and therapy system.

FIG. 2A illustrates a bronchoscopic robot in concert with a non-invasive histotripsy robot.

FIG. 2B illustrates a bronchoscopic robot in concert with a non-invasive histotripsy robot in a cone beam CT environment.

FIG. 3A illustrates a multi-approach for lung including a laparascopic/endoscopic robot and a separate histotripsy bed side robot cart.

FIG. 3B illustrates a laparascopic / endoscopic robot enabled with non-invasive histotripsy.

FIG. 4 illustrates a multi-approach for pancreas/liver resection that can be bed or patient side cart based.

### DETAILED DESCRIPTION

The system, methods and devices of the disclosure may be used for open surgical, minimally invasive surgical (laparascopic and percutaneous), robotic surgical (integrated into a robotically-enabled medical system), endoscopic or completely transdermal extracorporeal non-invasive acoustic cavitation for the treatment of healthy, diseased and/or injured tissue including but not limited to tissue destruction, cutting, skeletonizing and ablation. Furthermore, due to tissue selective properties, histotripsy may be used to create a cytoskeleton that allows for subsequent tissue regeneration either de novo or through the application of stem cells and other adjuvants. Finally, histotripsy can be used to cause the release of delivered agents such as chemotherapy and immunotherapy by locally causing the release of these agents by the application of acoustic energy to the targets. As will be described below, the acoustic cavitation system may include various sub-systems, including a Cart, Therapy, Integrated Imaging,

Robotics, Coupling and Software. The system also may comprise various Other Components, Ancillaries and Accessories, including but not limited to computers, cables and connectors, networking devices, power supplies, displays, drawers/storage, doors, wheels, and various simulation and training tools, etc. All systems, methods and means creating/controlling/delivering histotripsy are considered to be a part of this disclosure, including new related inventions disclosed herein.

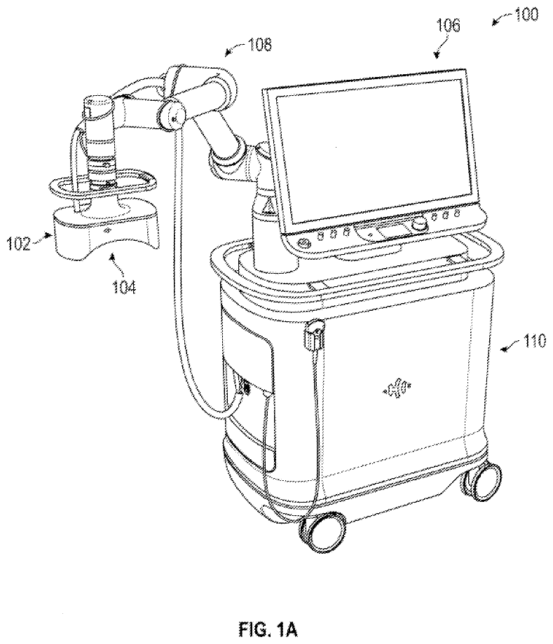

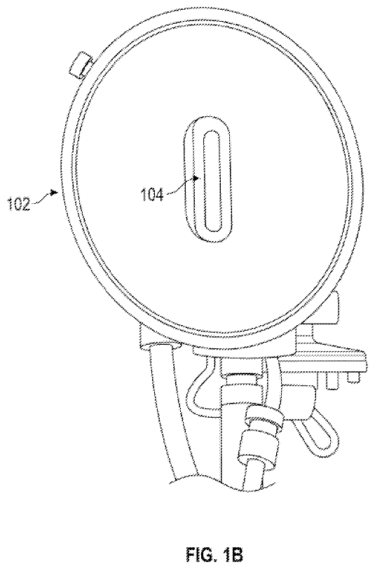

FIG. 1A generally illustrates histotripsy system 100 according to the present disclosure, comprising a therapy transducer 102, an imaging system 104, a display and control panel 106, a robotic positioning arm 108, and a cart 110. The system can further include an ultrasound coupling interface and a source of coupling medium, not shown.

FIG. 1B is a bottom view of the therapy transducer 102 and the imaging system 104. As shown, the imaging system can be positioned in the center of the therapy transducer. However, other embodiments can include the imaging system positioned in other locations within the therapy transducer, or even directly integrated into the therapy transducer. In some embodiments, the imaging system is configured to produce real-time imaging at a focal point of the therapy transducer. The system also allows for multiple imaging transducers to be located within the therapy transducer to provide multiple views of the target tissue simultaneously and to integrate these images into a single 3-D image.

The histotripsy system may comprise one or more of various sub-systems, including a Therapy sub-system that can create, apply, focus and deliver acoustic cavitation/histotripsy through one or more therapy transducers, Integrated Imaging sub-system (or connectivity to) allowing real-time visualization of the treatment site and histotripsy effect through-out the procedure, a Robotics positioning sub-system to mechanically and/or electronically steer the therapy transducer, further enabled to connect/support or interact with a Coupling sub-system to allow acoustic coupling between the therapy transducer and the patient, and Software to communicate, control and interface with the system and computer-based control systems (and other external systems) and various Other Components, Ancillaries and Accessories, including one or more user interfaces and displays, and related guided work-flows, all working in part or together. The system may further comprise various fluidics and fluid management components, including but not limited to, pumps, valve and flow controls, temperature and degassing controls, and irrigation and aspiration capabilities, as well as providing and storing fluids. It may also contain various power supplies and protectors.

### Cart

The Cart 110 may be generally configured in a variety of ways and form factors based on the specific uses and procedures. In some cases, systems may comprise multiple Carts, configured with similar or different arrangements. In some embodiments, the cart may be configured and arranged to be used in a radiology environment and in some cases in concert with imaging (e.g., CT, cone beam CT and/or MRI scanning). In other embodiments, it may be arranged for use in an operating room and a sterile environment for open surgical or laparascopic surgical and endoscopic application, or in a robotically enabled operating room, and used alone, or as part of a surgical robotics procedure wherein a surgical robot conducts specific tasks before, during or after use of the system and delivery of acoustic cavitation/histotripsy. As such and depending on the procedure environment based on the aforementioned embodiments, the cart may be positioned to provide sufficient work-space and access to various anatomical locations on the patient (e.g., torso, abdomen, flank, head and neck, etc.), as well as providing work-space for other systems (e.g., anesthesia cart, laparascopic tower, surgical robot, endoscope tower, etc.).

The Cart may also work with a patient surface (e.g., table or bed) to allow the patient to be presented and repositioned in a plethora of positions, angles and orientations, including allowing changes to such to be made pre, peri and post-procedurally. It may further comprise the ability to interface and communicate with one or more external imaging or image data management and communication systems, not limited to ultrasound, CT, fluoroscopy, cone beam CT, PET, PET/CT, MRI, optical, ultrasound, and image fusion and or image flow, of one or more modalities, to support the procedures and/or environments of use, including physical/mechanical interoperability (e.g., compatible within cone beam CT work-space for collecting imaging data pre, peri and/or post histotripsy) and to provide access to and display of patient medical data including but not limited to laboratory and historical medical record data.

In some embodiments one or more Carts may be configured to work together. As an example, one Cart may comprise a bedside mobile Cart equipped with one or more Robotic arms enabled with a Therapy transducer, and Therapy generator/amplifier, etc., while a companion cart working in concert and at a distance of the patient may comprise Integrated Imaging and a console/display for controlling the Robotic and Therapy facets, analogous to a surgical robot and master/slave configurations.

In some embodiments, the system may comprise a plurality of Carts, all slave to one master Cart, equipped to conduct acoustic cavitation procedures. In some arrangements and cases, one Cart configuration may allow for storage of specific sub-systems at a distance reducing operating room clutter, while another in concert Cart may comprise essentially bedside sub-systems and componentry (e.g., delivery system and therapy).

One can envision a plethora of permutations and configurations of Cart design, and these examples are in no way limiting the scope of the disclosure.

### Histotripsy

Histotripsy comprises short, high amplitude, focused ultrasound pulses to generate a dense, energetic, “bubble cloud”, capable of the targeted fractionation and destruction of tissue. Histotripsy is capable of creating controlled tissue erosion when directed at a tissue interface, including tissue/fluid interfaces, as well as well-demarcated tissue fractionation and destruction, at sub-cellular levels, when it is targeted at bulk tissue. Unlike other forms of ablation, including thermal and radiation-based modalities, histotripsy does not rely on heat cold or ionizing (high) energy to treat tissue. Instead, histotripsy uses acoustic cavitation generated at the focus to mechanically effect tissue structure, and in some cases liquefy, suspend, solubilize and/or destruct tissue into sub-cellular components.

Histotripsy can be applied in various forms, including: 1) Intrinsic-Threshold Histotripsy: Delivers pulses with a 1-2 cycles of high amplitude negative/tensile phase pressure exceeding the intrinsic threshold to generate cavitation in the medium (e.g., ˜24-28 MPa for water-based soft tissue), 2) Shock-Scattering Histotripsy: Delivers typically pulses 3-20 cycles in duration. The shockwave (positive/compressive phase) scattered from an initial individual microbubble generated forms inverted shockwave, which constructively interfere with the incoming negative/tensile phase to form high amplitude negative/rarefactional phase exceeding the intrinsic threshold. In this way, a cluster of cavitation microbubbles is generated. The amplitude of the tensile phases of the pulses is sufficient to cause bubble nuclei in the medium to undergo inertial cavitation within the focal zone throughout the duration of the pulse. These nuclei scatter the incident shockwaves, which invert and constructively interfere with the incident wave to exceed the threshold for intrinsic nucleation, and 3) Boiling Histotripsy: Employs pulses roughly 1-20 ms in duration. Absorption of the shocked pulse rapidly heats the medium, thereby reducing the threshold for intrinsic nuclei. Once this intrinsic threshold coincides with the peak negative pressure of the incident wave, boiling bubbles form at the focus.

The large pressure generated at the focus causes a cloud of acoustic cavitation bubbles to form above certain thresholds, which creates localized stress and strain in the tissue and mechanical breakdown without significant heat deposition. At pressure levels where cavitation is not generated, minimal effect is observed on the tissue at the focus. This cavitation effect is observed only at pressure levels significantly greater than those which define the inertial cavitation threshold in water for similar pulse durations, on the order of 10 to 30 MPa peak negative pressure.

Histotripsy may be performed in multiple ways and under different parameters. It may be performed totally non-invasively by acoustically coupling a focused ultrasound transducer over the skin of a patient and transmitting acoustic pulses transcutaneously through overlying (and intervening) tissue to the focal zone (treatment zone and site). The application of histotripsy is not limited to a transdermal approach but can be applied through any means that allows contact of the transducer with tissue including open surgical laparascopic surgical, percutaneous and robotically mediated surgical procedures. It may be further targeted, planned, directed and observed under direct visualization, via ultrasound imaging, given the bubble clouds generated by histotripsy may be visible as highly dynamic, echogenic regions on, for example, B Mode ultrasound images, allowing continuous visualization through its use (and related procedures). Likewise, the treated and fractionated tissue shows a dynamic change in echogenicity (typically a reduction), which can be used to evaluate, plan, observe and monitor treatment.

Generally, in histotripsy treatments, ultrasound pulses with 1 or more acoustic cycles are applied, and the bubble cloud formation relies on the pressure release scattering of the positive shock fronts (sometimes exceeding 100 MPa, P+) from initially initiated, sparsely distributed bubbles (or a single bubble). This is referred to as the “shock scattering mechanism”.

This mechanism depends on one (or a few sparsely distributed) bubble(s) initiated with the initial negative half cycle(s) of the pulse at the focus of the transducer. A cloud of microbubbles then forms due to the pressure release backscattering of the high peak positive shock fronts from these sparsely initiated bubbles. These back-scattered high-amplitude rarefactional waves exceed the intrinsic threshold thus producing a localized dense bubble cloud. Each of the following acoustic cycles then induces further cavitation by the backscattering from the bubble cloud surface, which grows towards the transducer. As a result, an elongated dense bubble cloud growing along the acoustic axis opposite the ultrasound propagation direction is observed with the shock scattering mechanism. This shock scattering process makes the bubble cloud generation not only dependent on the peak negative pressure, but also the number of acoustic cycles and the amplitudes of the positive shocks. Without at least one intense shock front developed by nonlinear propagation, no dense bubble clouds are generated when the peak negative half-cycles are below the intrinsic threshold.

When ultrasound pulses less than 2 cycles are applied, shock scattering can be minimized, and the generation of a dense bubble cloud depends on the negative half cycle(s) of the applied ultrasound pulses exceeding an “intrinsic threshold” of the medium. This is referred to as the “intrinsic threshold mechanism”.

This threshold can be in the range of 26-30 MPa for soft tissues with high water content, such as tissues in the human body. In some embodiments, using this intrinsic threshold mechanism, the spatial extent of the lesion may be well-defined and more predictable. With peak negative pressures (P−) not significantly higher than this threshold, sub-wavelength reproducible lesions as small as half of the −6 dB beam width of a transducer may be generated.

With high-frequency Histotripsy pulses, the size of the smallest reproducible lesion becomes smaller, which is beneficial in applications that require precise lesion generation. However, high-frequency pulses are more susceptible to attenuation and aberration, rendering problematical treatments at a larger penetration depth (e.g., ablation deep in the body) or through a highly aberrative medium (e.g., transcranial procedures, or procedures in which the pulses are transmitted through bone(s)). Histotripsy may further also be applied as a low-frequency “pump” pulse (typically <2 cycles and having a frequency between 100 kHz and 1 MHz) can be applied together with a high-frequency “probe” pulse (typically <2 cycles and having a frequency greater than 2 MHz, or ranging between 2 MHz and 10 MHz) wherein the peak negative pressures of the low and high-frequency pulses constructively interfere to exceed the intrinsic threshold in the target tissue or medium. The low-frequency pulse, which is more resistant to attenuation and aberration, can raise the peak negative pressure P− level for a region of interest (ROI), while the high-frequency pulse, which provides more precision, can pin-point a targeted location within the ROI and raise the peak negative pressure P− above the intrinsic threshold. This approach may be referred to as “dual frequency”, “dual beam histotripsy” or “parametric histotripsy.”

Additional systems, methods and parameters to deliver optimized histotripsy, using shock scattering, intrinsic threshold, and various parameters enabling frequency compounding and bubble manipulation, are herein included as part of the system and methods disclosed herein, including additional means of controlling said histotripsy effect as pertains to steering and positioning the focus, and concurrently managing tissue effects (e.g., prefocal thermal collateral damage) at the treatment site or within intervening tissue. Further, it is disclosed that the various systems and methods, which may include a plurality of parameters, such as but not limited to, frequency, operating frequency, center frequency, pulse repetition frequency, pulses, bursts, number of pulses, cycles, length of pulses, amplitude of pulses, pulse period, delays, burst repetition frequency, sets of the former, loops of multiple sets, loops of multiple and/or different sets, sets of loops, and various combinations or permutations of, etc., are included as a part of this disclosure, including future envisioned embodiments of such.

### Therapy Components

The Therapy sub-system may work with other sub-systems to create, optimize, deliver, visualize, monitor and control acoustic cavitation, also referred to herein and in following as “histotripsy”, and its derivatives of, including boiling histotripsy and other thermal high frequency ultrasound approaches. It is noted that the disclosed inventions may also further benefit other acoustic therapies that do not comprise a cavitation, mechanical or histotripsy component. The therapy sub-system can include, among other features, an ultrasound therapy transducer and a pulse generator system configured to deliver ultrasound pulses into tissue.

In order to create and deliver histotripsy and derivatives of histotripsy, the therapy sub-system may also comprise components, including but not limited to, one or more function generators, amplifiers, therapy transducers and power supplies.

The therapy transducer can comprise a single element or multiple elements configured to be excited with high amplitude electric pulses (>1000V or any other voltage that can cause harm to living organisms). The amplitude necessary to drive the therapy transducers for Histotripsy vary depending on the design of the transducer and the materials used (e.g., solid or polymer/piezoelectric composite including ceramic or single crystal) and the transducer center frequency which is directly proportional to the thickness of the piezoelectric material.

Transducers therefore operating at a high frequency require lower voltage to produce a given surface pressure than is required by low frequency therapy transducers. In some embodiments, the transducer elements are formed using a piezoelectric-polymer composite material or a solid piezoelectric material. Further, the piezoelectric material can be of polycrystalline/ceramic or single crystalline formulation. In some embodiments the transducer elements can be formed using silicon using MEMs technology, including CMUT and PMUT designs.

In some embodiments, the function generator may comprise a field programmable gate array (FPGA) or other suitable function generator. The FPGA may be configured with parameters disclosed previously herein, including but not limited to frequency, pulse repetition frequency, bursts, burst numbers, where bursts may comprise pulses, numbers of pulses, length of pulses, pulse period, delays, burst repetition frequency or period, where sets of bursts may comprise a parameter set, where loop sets may comprise various parameter sets, with or without delays, or varied delays, where multiple loop sets may be repeated and/or new loop sets introduced, of varied time delay and independently controlled, and of various combinations and permutations of such, overall and throughout.

In some embodiments, the generator or amplifier may be configured to be a universal single-cycle or multi-cycle pulse generator, and to support driving via Class D or inductive driving, as well as across all envisioned clinical applications, use environments, also discussed in part later in this disclosure. In other embodiments, the class D or inductive current driver may be configured to comprise transformer and/or auto-transformer driving circuits to further provide step up/down components, and in some cases, to preferably allow a step up in the amplitude. They may also comprise specific protective features, to further support the system, and provide capability to protect other parts of the system (e.g., therapy transducer and/or amplifier circuit components) and/or the user, from various hazards, including but not limited to, electrical safety hazards, which may potentially lead to use environment, system and therapy system, and user harms, damage or issues.

Disclosed generators may allow and support the ability of the system to select, vary and control various parameters (through enabled software tools), including, but not limited to those previously disclosed, as well as the ability to start/stop therapy, set and read voltage level, pulse and/or burst repetition frequency, number of cycles, duty ratio, channel enabled and delay, etc., modulate pulse amplitude on a fast time-scale independent of a high voltage supply, and/or other service, diagnostic or treatment features.

In some embodiments, the Therapy sub-system and/or components of, such as the amplifier, may comprise further integrated computer processing capability and may be networked, connected, accessed, and/or be removable/portable, modular, and/or exchangeable between systems, and/or driven/commanded from/by other systems, or in various combinations. Other systems may include other acoustic cavitation/histotripsy, HIFU, HITU, radiation therapy, radiofrequency, microwave, and cryoablation systems, navigation and localization systems, open surgical, laparascopic, single incision/single port, endoscopic and non-invasive surgical robots, laparascopic or surgical towers comprising other energy-based or vision systems, surgical system racks or booms, imaging carts, etc.

In some embodiments, one or more amplifiers may comprise a Class D amplifier and related drive circuitry including matching network components. Depending on the transducer element electric impedance and choice of the matching network components (e.g., an LC circuit made of an inductor L1 in series and the capacitor C1 in parallel), the combined impedance can be aggressively set low in order to have high amplitude electric waveform necessary to drive the transducer element. The maximum amplitude that Class D amplifiers is dependent on the circuit components used, including the driving MOSFET/IGBT transistors, matching network components or inductor, and transformer or autotransformer, and of which may be typically in the low kV (e.g., 1-3 kV) range.

Therapy transducer element(s) are excited with an electrical waveform with an amplitude (voltage) to produce a pressure output sufficient for Histotripsy therapy. The excitation electric field can be defined as the necessary waveform voltage per thickness of the piezoelectric element. For example, because a piezoelectric element operating at 1 MHz transducer is half the thickness of an equivalent 500 kHz element, it will require half the voltage to achieve the same electric field and surface pressure.

The Therapy sub-system may also comprise therapy transducers of various designs and working parameters, supporting use in various procedures (and procedure settings). Systems may be configured with one or more therapy transducers, that may be further interchangeable, and work with various aspects of the system in similar or different ways (e.g., may interface to a robotic arm using a common interface and exchange feature, or conversely, may adapt to work differently with application specific imaging probes, where different imaging probes may interface and integrate with a therapy transducer in specifically different ways).

Therapy transducers may be configured of various parameters that may include size, shape (e.g., rectangular or round; anatomically curved housings, etc.), geometry, focal length, number of elements, size of elements, distribution of elements (e.g., number of rings, size of rings for annular patterned transducers), frequency, enabling electronic beam steering, etc. Transducers may be composed of various materials (e.g., piezoelectric, silicon, etc.), form factors and types (e.g., machined elements, chip-based, etc.) and/or by various methods of fabrication of.

Transducers may be designed and optimized for clinical applications (e.g., abdominal tumors, peripheral vascular disease, fat ablation, etc.) and desired outcomes (e.g., acoustic cavitation/histotripsy without thermal injury to intervening tissue), and affording a breadth of working ranges, including relatively shallow and superficial targets (e.g., thyroid or breast nodules), versus, deeper or harder to reach targets, such as central liver or brain tumors. They may be configured to enable acoustic cavitation/histotripsy under various parameters and sets of, as enabled by the aforementioned system components (e.g., function generator and amplifier, etc.), including but not limited to frequency, pulse repetition rate, pulses, number of pulses, pulse length, pulse period, delays, repetitions, sync delays, sync period, sync pulses, sync pulse delays, various loop sets, others, and permutations of. The transducer may also be designed to allow for the activation of a drug payload either deposited in tissue through various means including injection, placement or delivery in micelle or nanostructures.

### Integrated Imaging

The disclosed system may comprise various imaging modalities to allow users to visualize, monitor and collect/use feedback of the patient's anatomy, related regions of interest and treatment/procedure sites, as well as surrounding and intervening tissues to assess, plan and conduct procedures, and adjust treatment parameters as needed. Imaging modalities may comprise various ultrasound, x-ray, CT, MRI, PET, fluoroscopy, optical, contrast or agent enhanced versions, and/or various combinations of. It is further disclosed that various image processing and characterization technologies may also be utilized to afford enhanced visualization and user decision making. These may be selected or commanded manually by the user or in an automated fashion by the system. The system may be configured to allow side by side, toggling, overlays, 3D reconstruction, segmentation, registration, multi-modal image fusion, image flow, and/or any methodology affording the user to identify, define and inform various aspects of using imaging during the procedure, as displayed in the various system user interfaces and displays. Examples may include locating, displaying and characterizing regions of interest, organ systems, potential treatment sites within, with on and/or surrounding organs or tissues, identifying critical structures such as ducts, vessels, nerves, ureters, fissures, capsules, tumors, tissue trauma/injury/disease, other organs, connective tissues, etc., and/or in context to one another, of one or more (e.g., tumor draining lymphatics or vasculature; or tumor proximity to organ capsule or underlying other organ), as unlimited examples.

Systems may be configured to include onboard integrated imaging hardware, software, sensors, probes and wetware, and/or may be configured to communicate and interface with external imaging and image processing systems. The aforementioned components may be also integrated into the system's Therapy sub-system components wherein probes, imaging arrays, or the like, and electrically, mechanically or electromechanically integrated into therapy transducers. This may afford, in part, the ability to have geometrically aligned imaging and therapy, with the therapy directly within the field of view, and in some cases in line, with imaging. In some embodiments, this integration may comprise a fixed orientation of the imaging capability (e.g., imaging probe) in context to the therapy transducer. In other embodiments, the imaging solution may be able to move or adjust its position, including modifying angle, extension (e.g., distance from therapy transducer or patient), rotation (e.g., imaging plane in example of an ultrasound probe) and/or other parameters, including moving/adjusting dynamically while actively imaging. The imaging component or probe may be encoded so its orientation and position relative to another aspect of the system, such as the therapy transducer, and/or robotically-enabled positioning component may be determined.

In one embodiment, the system may comprise onboard ultrasound, further configured to allow users to visualize, monitor and receive feedback for procedure sites through the system displays and software, including allowing ultrasound imaging and characterization (and various forms of), ultrasound guided planning and ultrasound guided treatment, all in real-time. The system may be configured to allow users to manually, semi-automated or in fully automated means image the patient (e.g., by hand or using a robotically-enabled imager).

In some embodiments, imaging feedback and monitoring can include monitoring changes in: backscatter from bubble clouds; speckle reduction in backscatter; backscatter speckle statistics; mechanical properties of tissue (i.e., elastography); tissue perfusion (i.e., ultrasound contrast); shear wave propagation; acoustic emissions, electrical impedance tomography, and/or various combinations of, including as displayed or integrated with other forms of imaging (e.g., CT or MRI).

In some embodiments, imaging including feedback and monitoring from backscatter from bubble clouds, may be used as a method to determine immediately if the histotripsy process has been initiated, is being properly maintained, or even if it has been extinguished. For example, this method enables continuously monitored in real time drug delivery, tissue erosion, and the like. The method also can provide feedback permitting the histotripsy process to be initiated at a higher intensity and maintained at a much lower intensity. For example, backscatter feedback can be monitored by any transducer or ultrasonic imager. By measuring feedback for the therapy transducer, an accessory transducer can send out interrogation pulses or be configured to passively detect cavitation. Moreover, the nature of the feedback received can be used to adjust acoustic parameters (and associated system parameters) to optimize the drug delivery and/or tissue erosion process.

In some embodiments, imaging including feedback and monitoring from backscatter, and speckle reduction, may be configured in the system.

For systems comprising feedback and monitoring via backscattering, and as means of background, as tissue is progressively mechanically subdivided, in other words homogenized, disrupted, or eroded tissue, this process results in changes in the size and distribution of acoustic scatter. At some point in the process, the scattering particle size and density is reduced to levels where little ultrasound is scattered, or the amount scattered is reduced significantly. This results in a significant reduction in speckle, which is the coherent constructive and destructive interference patterns of light and dark spots seen on images when coherent sources of illumination are used; in this case, ultrasound. After some treatment time, the speckle reduction results in a dark area in the therapy volume. Since the amount of speckle reduction is related to the amount of tissue subdivision, it can be related to the size of the remaining tissue fragments. When this size is reduced to sub-cellular levels, no cells are assumed to have survived. So, treatment can proceed until a desired speckle reduction level has been reached. Speckle is easily seen and evaluated on standard ultrasound imaging systems. Specialized transducers and systems, including those disclosed herein, may also be used to evaluate the backscatter changes.

Further, systems comprising feedback and monitoring via speckle, and as means of background, an image may persist from frame to frame and change very little as long as the scatter distribution does not change and there is no movement of the imaged object. However, long before the scatters are reduced enough in size to cause speckle reduction, they may be changed sufficiently to be detected by signal processing and other means. This family of techniques can operate as detectors of speckle statistics changes. For example, the size and position of one or more speckles in an image will begin to decorrelate before observable speckle reduction occurs. Speckle decorrelation, after appropriate motion compensation, can be a sensitive measure of the mechanical disruption of the tissues, and thus a measure of therapeutic efficacy. This feedback and monitoring technique may permit early observation of changes resulting from the acoustic cavitation/histotripsy process and can identify changes in tissue before substantial or complete tissue effect (e.g., erosion occurs). In one embodiment, this method may be used to monitor the acoustic cavitation/histotripsy process for enhanced drug delivery where treatment sites/tissue is temporally disrupted, and tissue damage/erosion is not desired. In other embodiments, this may comprise speckle decorrelation by movement of scatters in an increasingly fluidized therapy volume. For example, in the case where partial or complete tissue erosion is desired.

For systems comprising feedback and monitoring via elastography, and as means of background, as treatment sites/tissue are further subdivided per an acoustic cavitation/histotripsy effect (homogenized, disrupted, or eroded), its mechanical properties change from a soft but interconnected solid to a viscous fluid or paste with few long-range interactions. These changes in mechanical properties can be measured by various imaging modalities including MRI and ultrasound imaging systems. For example, an ultrasound pulse can be used to produce a force (i.e., a radiation force) on a localized volume of tissue. The tissue response (displacements, strains, and velocities) can change significantly during histotripsy treatment allowing the state of tissue disruption to be determined by imaging or other quantitative means.

Systems may also comprise feedback and monitoring via shear wave propagation changes. As means of background, the subdivision of tissues makes the tissue more fluid and less solid and fluid systems generally do not propagate shear waves. Thus, the extent of tissue fluidization provides opportunities for feedback and monitoring of the histotripsy process. For example, ultrasound and MRI imaging systems can be used to observe the propagation of shear waves. The extinction of such waves in a treated volume is used as a measure of tissue destruction or disruption. In one system embodiment, the system and supporting sub-systems may be used to generate and measure the interacting shear waves. For example, two adjacent ultrasound foci might perturb tissue by pushing it in certain ways. If adjacent foci are in a fluid, no shear waves propagate to interact with each other. If the tissue is not fluidized, the interaction would be detected with external means, for example, by a difference frequency only detected when two shear waves interact nonlinearly, with their disappearance correlated to tissue damage. As such, the system may be configured to use this modality to enhance feedback and monitoring of the acoustic cavitation/histotripsy procedure.

For systems comprising feedback and monitoring via acoustic emission, and as means of background, as a tissue volume is subdivided, its effect on acoustic cavitation/histotripsy (e.g., the bubble cloud here) is changed. For example, bubbles may grow larger and have a different lifetime and collapse changing characteristics in intact versus fluidized tissue. Bubbles may also move and interact after tissue is subdivided producing larger bubbles or cooperative interaction among bubbles, all of which can result in changes in acoustic emission. These emissions can be heard during treatment and they change during treatment. Analysis of these changes, and their correlation to therapeutic efficacy, enables monitoring of the progress of therapy, and may be configured as a feature of the system.

For systems comprising feedback and monitoring via electrical impedance tomography, and as means of background, an impedance map of a therapy site can be produced based upon the spatial electrical characteristics throughout the therapy site. Imaging of the conductivity or permittivity of the therapy site of a patient can be inferred from taking skin surface electrical measurements. Conducting electrodes are attached to a patient's skin and small alternating currents are applied to some or all of the electrodes. One or more known currents are injected into the surface and the voltage is measured at a number of points using the electrodes.

The process can be repeated for different configurations of applied current. The resolution of the resultant image can be adjusted by changing the number of electrodes employed. A measure of the electrical properties of the therapy site within the skin surface can be obtained from the impedance map, and changes in and location of the acoustic cavitation/histotripsy (e.g., bubble cloud, specifically) and histotripsy process can be monitored using this as configured in the system and supporting sub-systems.

The user may be allowed to further select, annotate, mark, highlight, and/or contour, various regions of interest or treatment sites, and defined treatment targets (on the image(s)), of which may be used to command and direct the system where to image, test and/or treat, through the system software and user interfaces and displays. In some arrangements, the user may use a manual ultrasound probe (e.g., diagnostic hand-held probe) to conduct the procedure. In another arrangement, the system may use a robot and/or electromechanical positioning system to conduct the procedure, as directed and/or automated by the system, or conversely, the system can enable combinations of manual and automated uses.

The system may further include the ability to conduct image registration, including imaging and image data set registration to allow navigation and localization of the system to the patient, including the treatment site (e.g., tumor, critical structure, bony anatomy, anatomy and identifying features of, etc.). In one embodiment, the system allows the user to image and identify a region of interest, for example the liver, using integrated ultrasound, and to select and mark a tumor (or surrogate marker of) comprised within the liver through/displayed in the system software, and wherein said system registers the image data to a coordinate system defined by the system, that further allows the system's Therapy and Robotics sub-systems to deliver synchronized acoustic cavitation/histotripsy to said marked tumor. The system may comprise the ability to register various image sets, including those previously disclosed, to one another, as well as to afford navigation and localization (e.g., of a therapy transducer to a CT or MRI/ultrasound fusion image with the therapy transducer and Robotics sub-system tracking to said image).

The system may also comprise the ability to work in a variety of interventional, endoscopic and surgical environments, including alone and with other systems (surgical/laparascopic towers, vision systems, endoscope systems and towers, ultrasound enabled endoscopic ultrasound (flexible and rigid), percutaneous/endoscopic/laparascopic and minimally invasive navigation systems (e.g., optical, electromagnetic, shape-sensing, ultrasound-enabled, etc.), of also which may work with, or comprise various optical imaging capabilities (e.g., fiber and or digital). The disclosed system may be configured to work with these systems, in some embodiments working alongside them in concert, or in other embodiments where all or some of the system may be integrated into the above systems/platforms (e.g., acoustic cavitation/histotripsy-enabled endoscope system or laparascopic surgical robot). In many of these environments, a therapy transducer may be utilized at or around the time of use, for example, of an optically guided endoscope/bronchoscope, or as another example, at the time a laparascopic robot (e.g., Intuitive Da Vinci* Xi system) is viewing/manipulating a tissue/treatment site. Further, these embodiments and examples may include where said other systems/platforms are used to deliver (locally) fluid to enable the creation of a man-made acoustic window, where on under normal circumstances may not exist (e.g., fluidizing a segment or lobe of the lung in preparation for acoustic cavitation/histotripsy via non-invasive transthoracic treatment (e.g., transducer externally placed on/around patient). Systems disclosed herein may also comprise all or some of their sub-system hardware packaged within the other system cart/console/systems described here (e.g., acoustic cavitation/histotripsy system and/or sub-systems integrated and operated from said navigation or laparascopic system).

The system may also be configured, through various aforementioned parameters and other parameters, to display real-time visualization of a bubble cloud in a spatial-temporal manner, including the resulting tissue effect peri/post-treatment from tissue/bubble cloud interaction, wherein the system can dynamically image and visualize, and display, the bubble cloud, and any changes to it (e.g., decreasing or increasing echogenicity), which may include intensity, shape, size, location, morphology, persistence, etc. These features may allow users to continuously track and follow the treatment in real-time in one integrated procedure and interface/system, and confirm treatment safety and efficacy on the fly (versus other interventional or surgical modalities, which either require multiple procedures to achieve the same, or where the treatment effect is not visible in real-time (e.g., radiation therapy), or where it is not possible to achieve such (e.g., real-time visualization of local tissue during thermal ablation), and/or where the other procedure further require invasive approaches (e.g., incisions or punctures) and iterative imaging in a scanner between procedure steps (e.g., CT or MRI scanning). The above disclosed systems, sub-systems, components, modalities, features and work-flows/methods of use may be implemented in an unlimited fashion through enabling hardware, software, user interfaces and use environments, and future improvements, enhancements and inventions in this area are considered as included in the scope of this disclosure, as well as any of the resulting data and means of using said data for analytics, artificial intelligence or digital health applications and systems.

### Robotics

They system may comprise various Robotic sub-systems and components, including but not limited to, one or more robotic arms and controllers, which may further work with other sub-systems or components of the system to deliver and monitor acoustic cavitation/histotripsy. As previously discussed herein, robotic arms and control systems may be integrated into one or more Cart configurations.

For example, one system embodiment may comprise a Cart with an integrated robotic arm and control system, and Therapy, Integrated Imaging and Software, where the robotic arm and other listed sub-systems are controlled by the user through the form factor of a single bedside Cart.

In other embodiments, the Robotic sub-system may be configured in one or more separate Carts, that may be a driven in a master/slave configuration from a separate master or Cart, wherein the robotically-enabled Cart is positioned bed/patient-side, and the Master is at a distance from said Cart.

Disclosed robotic arms may be comprised of a plurality of joints, segments, and degrees of freedom and may also include various integrated sensor types and encoders, implemented for various use and safety features. Sensing technologies and data may comprise, as an example, vision, potentiometers, position/localization, kinematics, force, torque, speed, acceleration, dynamic loading, and/or others. In some cases, sensors may be used for users to direct robot commands (e.g., hand gesture the robot into a preferred set up position, or to dock home). Additional details on robotic arms can be found in US Patent Pub. No. 2013/0255426 to Kassow et al. which is disclosed herein by reference in its entirety.

The robotic arm receives control signals and commands from the robotic control system, which may be housed in a Cart. The system may be configured to provide various functionalities, including but not limited to, position, tracking, patterns, triggering, and events/actions.

Position may be configured to comprise fixed positions, pallet positions, time-controlled positions, distance-controlled positions, variable-time controlled positions, variable- distance controlled positions.

Tracking may be configured to comprise time-controlled tracking and/or distance-controlled tracking.

The patterns of movement may be configured to comprise intermediate positions or waypoints, as well as sequence of positions, through a defined path in space.

Triggers may be configured to comprise distance measuring means, time, and/or various sensor means including those disclosed herein, and not limited to, visual/imaging-based, force, torque, localization, energy/power feedback and/or others.

Events/actions may be configured to comprise various examples, including proximity-based (approaching/departing a target object), activation or deactivation of various end-effectors (e.g., therapy transducers), starting/stopping/pausing sequences of said events, triggering or switching between triggers of events/actions, initiating patterns of movement and changing/toggling between patterns of movement, and/or time-based and temporal over the defined work and time-space.

In one embodiment, the system comprises a three degree of freedom robotic positioning system, enabled to allow the user (through the software of the system and related user interfaces), to micro-position a therapy transducer through X, Y, and Z coordinate system, and where gross macro-positioning of the transducer (e.g., aligning the transducer on the patient's body) is completed manually. In some embodiments, the robot may comprise 6 degrees of freedom including X, Y, Z, and pitch, roll and yaw. In other embodiments, the Robotic sub-system may comprise further degrees of freedom, that allow the robot arm supporting base to be positioned along a linear axis running parallel to the general direction of the patient surface, and/or the supporting base height to be adjusted up or down, allowing the position of the robotic arm to be modified relative to the patient, patient surface, Cart, Coupling sub-system, additional robots/robotic arms and/or additional surgical systems, including but not limited to, surgical towers, imaging systems, endoscopic/laparascopic systems, and/or other.

One or more robotic arms may also comprise various features to assist in maneuvering and modifying the arm position, manually or semi-manually, and of which said features may interface on or between the therapy transducer and the most distal joint of the robotic arm. In some embodiments, the feature is configured to comprise a handle allowing maneuvering and manual control with one or more hands. The handle may also be configured to include user input and electronic control features of the robotic arm, to command various drive capabilities or modes, to actuate the robot to assist in gross or fine positioning of the arm (e.g., activating or deactivating free drive mode). The work-flow for the initial positioning of the robotic arm and therapy head can be configured to allow either first positioning the therapy transducer/head in the coupling solution, with the therapy transducer directly interfaced to the arm, or in a different work-flow, allowing the user to set up the coupling solution first, and enabling the robot arm to be interfaced to the therapy transducer/coupling solution as a later/terminal set up step.

In some embodiments, the robotic arm may comprise a robotic arm on a laparascopic, single port, endoscopic, hybrid or combination of, and/or other robot, wherein said robot of the system may be a slave to a master that controls said arm, as well as potentially a plurality of other arms, equipped to concurrently execute other tasks (vision, imaging, grasping, cutting, ligating, sealing, closing, stapling, ablating, suturing, marking, etc.), including actuating one or more laparascopic arms (and instruments) and various histotripsy system components. For example, a laparascopic robot may be utilized to prepare the surgical site, including manipulating organ position to provide more ideal acoustic access and further stabilizing said organ in some cases to minimize respiratory motion. In conjunction and parallel to this, a second robotic arm may be used to deliver non-invasive acoustic cavitation through a body cavity, as observed under real-time imaging from the therapy transducer (e.g., ultrasound) and with concurrent visualization via a laparascopic camera. In other related aspects, a similar approach may be utilized with a combination of an endoscopic and non-invasive approach, and further, with a combination of an endoscopic, laparascopic and non-invasive approach.

### Coupling

Systems may comprise a variety of Coupling sub-system embodiments, of which are enabled and configured to allow acoustic coupling to the patient to afford effective acoustic cavitation/histotripsy (e.g., provide acoustic medium between transducer and patient, and support of). These may include different form factors of such, including open and enclosed solutions, and some arrangements which may be configured to allow dynamic control over the acoustic medium (e.g., temperature, dissolved gas content, level of particulate filtration, sterility, etc.). Such dynamic control components may be directly integrated to the system (within the Cart), or may be in communication with the system, but externally situated.

The Coupling sub-system typically comprises, at a minimum, coupling medium, a reservoir/container to contain said coupling medium, and a support structure. In most embodiments, the coupling medium is water, and wherein the water may be conditioned before or during the procedure (e.g., chilled, degassed, filtered, etc.). Various conditioning parameters may be employed based on the configuration of the system and it's intended use/application.

The reservoir or medium container may be formed and shaped to adapt/conform to the patient, allow the therapy transducer to engage and work within the acoustic medium, per defined and required working space (minimum volume of medium to allow the therapy transducer to be positioned and/or move through one or more treatment positions or patterns, and at various standoffs or depths from the patient, etc.), and wherein said reservoir or medium container may also mechanically support the load, and distribution of the load, through the use of a mechanical and/or electromechanical support structure. The container may be of various shapes, sizes, curvatures, and dimensions, and may be comprised of a variety of materials (single, multiple, composites, etc.), of which may vary throughout. In some embodiments, it may comprise features such as films, drapes, membranes, bellows, etc. that may be insertable and removable, and/or fabricated within. It may further contain various sensors, drains, lighting (e.g., LEDs), markings, text, etc.

In one embodiment, the reservoir or medium container contains a sealable frame, of which a membrane and/or film may be positioned within, to afford a conformable means of contacting the reservoir (later comprising the therapy transducer) as an interface to the patient, that further provides a barrier to the medium (e.g., water) between the patient and transducer). In other embodiments, the membrane and/or film may comprise an opening, the edge of which affords mechanical sealing to the patient, but in contrast allows medium communication with the patient (e.g., direct water interface with patient). The superstructure of the reservoir or medium container in both these examples may further afford the proximal portion of the structure (e.g., top) to be open or enclosed (e.g., to prevent spillage or afford additional features).

Disclosed membranes may be comprised of various elastomers, viscoelastic polymers, thermoplastics, thermoplastic elastomers, thermoset polymers, silicones, urethanes, rigid/flexible co-polymers, block co-polymers, random block co-polymers, etc. Materials may be hydrophilic, hydrophobic, surface modified, coated, extracted, etc., and may also contain various additives to enhance performance, appearance or stability. In some embodiments, the thermoplastic elastomer may be styrene-ethylene-butylene-styrene (SEBS), or other like strong and flexible elastomers.

Said materials may be formed into useful membranes through molding, casting, spraying, ultrasonic spraying and/or any other processing methodology that produces useful embodiments. They may be single use or reposable/reusable. They may be provided non-sterile, aseptically cleaned or sterile, where sterilization may comprise any known method, including but not limited to ethylene oxide, gamma, e-beam, autoclaving, steam, peroxide, plasma, chemical, etc. Membranes can be further configured with an outer molded frame to provide mechanical stability during assembly of the coupling sub-system. Various parameters of the membrane can be optimized for this method of use, including thickness, thickness profile, density, formulation (e.g., polymer molecular weight and copolymer ratios), including optimizing specifically to maximize acoustic properties, including minimizing impact to cavitation initiation threshold values, and/or ultrasound imaging artifacts, including but not limited to membrane reflections.

Open reservoirs or medium containers may comprise various methods of filling, including using pre-prepared medium or water, that may be delivered into the such, in some cases to a defined specification of water (level of temperature and gas saturation, etc.), or they may comprise additional features integral to the design that allow filling and draining (e.g., ports, valves, hoses, tubing, fittings, bags, pumps, etc.).

Enclosed iterations of the reservoir or medium container may comprise various features for sealing, in some embodiments sealing to a proximal/top portion or structure of a reservoir/container, or in other cases where sealing may comprise embodiments that seal to the transducer, or a feature on the transducer housings. Further, some embodiments may comprise the dynamic ability to control the volume of fluid within these designs, to minimize the potential for air bubbles or turbulence in said fluid and to allow for changes in the focal length to the target area without moving the transducer. As such, integrated features allowing fluid communication, and control of, may be provided (ability to provide/remove fluid on demand), including the ability to monitor and control various fluid parameters, some disclosed above. In order to provide this functionality, the overall system, and as part, the Coupling sub-system, may comprise a fluid conditioning system, which may contain various electromechanical devices, systems, power, sensing, computing and control systems, etc. The reservoir may also be configured to receive signals that cause it to deform or change shape in a specific and controlled manner to allow the target point to be adjusted without moving the transducer.

Coupling support systems may include various mechanical support devices to interface the reservoir/container and medium to the patient, and the workspace (e.g., bed). In some embodiments, the support system comprises a mechanical arm with 3 or more degrees of freedom. Said arm may interface with one or more locations (and features) of the bed, including but not limited to, the frame, rails, customized rails or inserts, as well as one or more locations of the reservoir or container. The arm may be a feature implemented on one or more Carts, wherein Carts may be configured in various unlimited permutations, in some cases where a Cart only comprises the role of supporting and providing the disclosed support structure.

In some embodiments, the support structure and arm may be a robotically-enabled arm, implemented as a stand-alone Cart, or integrated into a Cart further comprising two or more system sub-systems, or where in the robotically-enabled arm is an arm of another robot, of interventional, surgical or other type, and may further comprise various user input features to actuate/control the robotic arm (e.g., positioning into/within coupling medium) and/or Coupling solution features (e.g., filling, draining, etc.).

### Software

The system may comprise various software applications, features and components which allow the user to interact, control and use the system for a plethora of clinical applications. The Software may communicate and work with one or more of the sub-systems, including but not limited to Therapy, Integrated Imaging, Robotics and Other Components, Ancillaries and Accessories of the system.

Overall, in no specific order of importance, the software may provide features and support to initialize and set up the system, service the system, communicate and import/export/store data, modify/manipulate/configure/control/command various settings and parameters by the user, mitigate safety and use-related risks, plan procedures, provide support to various configurations of transducers, robotic arms and drive systems, function generators and amplifier circuits/slaves, test and treatment ultrasound sequences, transducer steering and positioning (electromechanical and electronic beam steering, etc.), treatment patterns, support for imaging and imaging probes, manual and electromechanical/robotically-enabling movement of, imaging support for measuring/characterizing various dimensions within or around procedure and treatment sites (e.g., depth from one anatomical location to another, etc., pre-treatment assessments and protocols for measuring/characterizing in situ treatment site properties and conditions (e.g., acoustic cavitation/histotripsy thresholds and heterogeneity of), targeting and target alignment, calibration, marking/annotating, localizing/navigating, registering, guiding, providing and guiding through work-flows, procedure steps, executing treatment plans and protocols autonomously, autonomously and while under direct observation and viewing with real-time imaging as displayed through the software, including various views and viewports for viewing, communication tools (video, audio, sharing, etc.), troubleshooting, providing directions, warnings, alerts, and/or allowing communication through various networking devices and protocols. It is further envisioned that the software user interfaces and supporting displays may comprise various buttons, commands, icons, graphics, text, etc., that allow the user to interact with the system in a user-friendly and effective manner, and these may be presented in an unlimited number of permutations, layouts and designs, and displayed in similar or different manners or feature sets for systems that may comprise more than one display (e.g., touch screen monitor and touch pad), and/or may network to one or more external displays or systems (e.g., another robot, navigation system, system tower, console, monitor, touch display, mobile device, tablet, etc.).

The software, as a part of a representative system, including one or more computer processors, may support the various aforementioned function generators (e.g., FPGA), amplifiers, power supplies and therapy transducers. The software may be configured to allow users to select, determine and monitor various parameters and settings for acoustic cavitation/histotripsy, and upon observing/receiving feedback on performance and conditions, may allow the user to stop/start/modify said parameters and settings.

The software may be configured to allow users to select from a list or menu of multiple transducers and support the auto-detection of said transducers upon connection to the system (and verification of the appropriate sequence and parameter settings based on selected application). In other embodiments, the software may update the targeting and amplifier settings (e.g., channels) based on the specific transducer selection. The software may also provide transducer recommendations based on pre-treatment and planning inputs. Conversely, the software may provide error messages or warnings to the user if said therapy transducer, amplifier and/or function generator selections or parameters are erroneous, yield a fault or failure. This may further comprise reporting the details and location of such.

In addition to above, the software may be configured to allow users to select treatment sequences and protocols from a list or menu, and to store selected and/or previous selected sequences and protocols as associated with specific clinical uses or patient profiles.

Related profiles may comprise any associated patient, procedure, clinical and/or engineering data, and maybe used to inform, modify and/or guide current or future treatments or procedures/interventions, whether as decision support or an active part of a procedure itself (e.g., using serial data sets to build and guide new treatments).

As a part of planning or during the treatment, the software (and in working with other components of the system) may allow the user to evaluate and test acoustic cavitation/histotripsy thresholds at various locations in a user-selected region of interest or defined treatment area/volume, to determine the minimum cavitation thresholds throughout said region or area/volume, to ensure treatment parameters are optimized to achieve, maintain and dynamically control acoustic cavitation/histotripsy. In one embodiment, the system allows a user to manually evaluate and test threshold parameters at various points. Said points may include those at defined boundary, interior to the boundary and center locations/positions, of the selected region of interest and treatment area/volume, and where resulting threshold measurements may be reported/displayed to the user, as well as utilized to update therapy parameters before treatment. In another embodiment, the system may be configured to allow automated threshold measurements and updates, as enabled by the aforementioned Robotics sub-system, wherein the user may direct the robot, or the robot may be commanded to execute the measurements autonomously.

Software may also be configured, by working with computer processors and one or more function generators, amplifiers and therapy transducers, to allow various permutations of delivering and positioning optimized acoustic cavitation/histotripsy in and through a selected area/volume. This may include, but not limited to, systems configured with a fixed/natural focus arrangement using purely electromechanical positioning configuration(s), electronic beam steering (with or without electromechanical positioning), electronic beam steering to a new selected fixed focus with further electromechanical positioning, axial (Z axis) electronic beam steering with lateral (X and Y) electromechanical positioning, high speed axial electronic beam steering with lateral electromechanical positioning, high speed beam steering in 3D space, various combinations of including with dynamically varying one or more acoustic cavitation/histotripsy parameters based on the aforementioned ability to update treatment parameters based on threshold measurements (e.g., dynamically adjusting amplitude across the treatment area/volume).

### Other Components, Ancillaries and Accessories

The system may comprise various other components, ancillaries and accessories, including but not limited to computers, computer processors, power supplies including high voltage power supplies, controllers, cables, connectors, networking devices, software applications for security, communication, integration into information systems including hospital information systems, cellular communication devices and modems, handheld wired or wireless controllers, goggles or glasses for advanced visualization, augmented or virtual reality applications, cameras, sensors, tablets, smart devices, phones, internet of things enabling capabilities, specialized use “apps” or user training materials and applications (software or paper based), virtual proctors or trainers and/or other enabling features, devices, systems or applications, and/or methods of using the above.

### System Variations and Methods/Applications

In addition to performing a breadth of procedures, the system may allow additional benefits, such as enhanced planning, imaging and guidance to assist the user. In one embodiment, the system may allow a user to create a patient, target and application specific treatment plan, wherein the system may be configured to optimize treatment parameters based on feedback to the system during planning, and where planning may further comprise the ability to run various test protocols to gather specific inputs to the system and plan.

Feedback may include various energy, power, location, position, tissue and/or other parameters.

The system, and the above feedback, may also be further configured and used to autonomously (and robotically) execute the delivery of the optimized treatment plan and protocol, as visualized under real-time imaging during the procedure, allowing the user to directly observe the local treatment tissue effect, as it progresses through treatment, and start/stop/modify treatment at their discretion. Both test and treatment protocols may be updated over the course of the procedure at the direction of the user, or in some embodiments, based on logic embedded within the system.

It is also recognized that many of these benefits may further improve other forms of acoustic therapy, including thermal ablation with high intensity focused ultrasound (HIFU), high intensity therapeutic ultrasound (HITU) including boiling histotripsy (thermal cavitation), and are considered as part of this disclosure. The disclosure also considers the application of histotripsy as a means to activate previously delivered in active drug payloads whose activity is inert due to protection in a micelle, nanostructure or similar protective structure or through molecular arrangement that allows activation only when struck with acoustic energy.

In another aspect, the Therapy sub-system, comprising in part, one or more amplifiers, transducers and power supplies, may be configured to allow multiple acoustic cavitation and histotripsy driving capabilities, affording specific benefits based on application, method and/or patient specific use. These benefits may include, but are not limited to, the ability to better optimize and control treatment parameters, which may allow delivery of more energy, with more desirable thermal profiles, increased treatment speed and reduced procedure times, enable electronic beam steering and/or other features.

This disclosure also includes novel systems and concepts as related to systems and sub-systems comprising new and “universal” amplifiers, which may allow multiple driving approaches (e.g., single and multi-cycle pulsing). In some embodiments, this may include various novel features to further protect the system and user, in terms of electrical safety or other hazards (e.g., damage to transducer and/or amplifier circuitry).

In another aspect, the system, and Therapy sub-system, may include a plethora of therapy transducers, where said therapy transducers are configured for specific applications and uses and may accommodate treating over a wide range of working parameters (target size, depth, location, etc.) and may comprise a wide range of working specifications (detailed below). Transducers may further adapt, interface and connect to a robotically-enabled system, as well as the Coupling sub-system, allowing the transducer to be positioned within, or along with, an acoustic coupling device allowing, in many embodiments, concurrent imaging and histotripsy treatments through an acceptable acoustic window. The therapy transducer may also comprise an integrated imaging probe or localization sensors, capable of displaying and determining transducer position within the treatment site and affording a direct field of view (or representation of) the treatment site, and as the acoustic cavitation/histotripsy tissue effect and bubble cloud may or may not change in appearance and intensity, throughout the treatment, and as a function of its location within said treatment (e.g., tumor, healthy tissue surrounding, critical structures, adipose tissue, etc.).

The systems, methods and use of the system disclosed herein, may be beneficial to overcoming significant unmet needs in the areas of soft tissue ablation, oncology, immuno-oncology, advanced image guided procedures, surgical procedures including but not limited to open, laparascopic, single incision, natural orifice, endoscopic, non-invasive, various combination of, various interventional spaces for catheter-based procedures of the vascular, cardiovascular pulmonary and/or neurocranial-related spaces, cosmetics/aesthetics, metabolic (e.g., type 2 diabetes), plastic and reconstructive, ocular and ophthalmology, orthopedic, gynecology and men's health, and other systems, devices and methods of treating diseased, injured, undesired, or healthy tissues, organs or cells.

Systems and methods are also provided for improving treatment patterns within tissue that can reduce treatment time, improve efficacy, and reduce the amount of energy and prefocal tissue heating delivered to patients.

### Use Environments

The disclosed system, methods of use, and use of the system, may be conducted in a plethora of environments and settings, with or without various support systems such as anesthesia, including but not limited to, procedure suites, operating rooms, hybrid rooms, in and out-patient settings, ambulatory settings, imaging centers, radiology, radiation therapy, oncology, surgical and/or any medical center, as well as physician offices, mobile healthcare centers or systems, automobiles and related vehicles (e.g., van), aero and marine transportation vehicles such as planes and ships, and/or any structure capable of providing temporary procedure support (e.g., tent). In some cases, systems and/or sub-systems disclosed herein may also be provided as integrated features into other environments, for example, the direct integration of the histotripsy Therapy sub-system into a MRI scanner or patient surface/bed, wherein at a minimum the therapy generator and transducer are integral to such, and in other cases wherein the histotripsy configuration further includes a robotic positioning system, which also may be integral to a scanner or bed centered design.

### Multi-Approach Histotripsy Robotic Systems and Methods

Significant unmet needs exist in interventional and surgical medical procedures today, including those procedures utilizing minimally invasive devices and approaches to treat disease and/or injury, and across various types of procedures where the unmet needs may be solved with entirely new medical procedures. Today's medical system capabilities are often limited by access, wherein a less or non-invasive approach would be preferred, or wherein today's tools aren't capable to deliver preferred/required tissue effects (e.g., operate around/through critical structures without serious injury), or where the physical set up of the systems makes certain procedure approaches less desirable or not possible, and where a combination of approaches, along with enhanced tissue effecting treatments, may enable entirely new procedures and approaches, not possible today.

Disclosed herein, are robotic systems and methods, where both may utilize various combinations of percutaneous/laparascopic, endoscopic and/or non-invasive/transcutaneous devices, controlled through various combinations of manual, semi-manual and/or automated approaches, together enabled to allow the targeted delivery of histotripsy and acoustic cavitation, and where histotripsy may be either one step to enable further steps in the operation/procedure (e.g., treating tumor entangled around critical structures to convert inoperable patients to operable via robotic-assisted laparascopic resection), or conversely, where histotripsy is the main “treatment or therapy” intent of the procedure and the other robotic system of a multi-approach system/method is used to for other supporting procedure steps (e.g., visualization, ligation, fluidizing or stabilizing an organ or organ space). This may be fully enabled in a singular robotic platform architecture (e.g., a bed-based robot with arms configured for endoscopic and non-invasive approaches), or in contrast, this may be accomplished with multiple separate robotic architectures or systems, working in concert (e.g., an endoscopic bed-based surgical robot working with a collaborative non-invasive histotripsy cart-based robot).

As unlimited and representative examples to illustrate the disclosed concept, such systems and approaches may be used to enable pancreatic cancer resection, wherein a bedside non-invasive histotripsy robotic system may be used to treat tumor involved vasculature (which would normally render a surgical approach too risky), and of which, upon completion, a laparascopic robotic resection of pancreas (and associated tumor) may be completed using a master slave laparascopic robot (column based patient side cart, e.g., Da Vinci Xi System, Intuitive Surgical). Similar approaches may be utilized, using multiple robot system architectures, for other cancer related surgical procedures, including but not limited to, liver, kidney, lung, colorectal, and other complex procedures, where many patients would benefit from better enabling, down staging, or assisting, in some fashion, better surgical approaches, or to opt more patients into surgical procedures who normally would not qualify for medical reasons, and/or allow more providers/surgeons, to conduct such procedures.

### Multi-Approach Setup and Operation Perspectives

Further, disclosed multi-approach histotripsy robotic systems may be configured to allow various permutations of setup, environment and operation perspective (e.g., orientation of user to system, placement of robotic arms and patient). Such systems also comprise the unique feature of having a concurrent combination of instrument/device access (e.g., ports or trocars, endoscopes of various types, catheter access, etc.), including acoustic access (e.g., devices/methods/materials allowing acoustic coupling for ultrasound visualization and histotripsy delivery along a planned/desired acoustic pathway). It is envisioned that there are limitless configurations and combinations of set up and access.

### Robotic System Architectures

In terms of robotic system architectures which may comprise one or more type of the disclosed multi-approach robotic methods, system architectures may include bed-based, column-based, boom-based, cart-based, imaging bed or gantry-based and pod-based, and/other envisioned system architectures, and combinations of. Some embodiments may comprise multiple sets of systems (e.g., a plurality of carts) each enabled with a minimum of one robotic arm. In embodiments using mixed combinations of system architectures (e.g., column and cart-based), said combinations may be interconnected through various software and/or electrical connections and related communication protocols (e.g., enable one system software or access to controls over system features/parameters on the other architecture system), or may be working in concert, but independent (with no direct electronic, mechanical and/or software communication connection between them). The use of multiple systems (and architectures) may be configured to be coupled/connected through one or more cables, in effort to simplify and declutter the environment. Specific functionality may be provided in separate cabling and connectors, including power, optics, imaging, ultrasound imaging, hospital information systems, histotripsy therapy, mechanical and robotics controls, fluidics, and/or other support for other controls. Connectors/cables may also be positioned in various locations on the systems, including but not limited to, side panels, arms, control panels and user interfaces, displays, end-effectors, transducers, patient coupling devices, etc.

### Control Systems and User Interfaces

In terms of system controls for robotic/system controls systems, systems may be configured to include consoles, user interfaces, displays, touch displays and associated controls (physical and software) integral to each of the form factors above (local to the form factor, e.g., on the patient side cart), or may be configured to further include master/slave, control room and/or other tele-remote configurations which further afford users to control and interact with such systems from a distance via known communication methods.

### Robotic Arms for Multi-Approach Systems

The robotic arms of disclosed systems may comprise various architectures, arm bases, degrees of freedom, joints, reach, payload capacity, repeatability, sensing capability, including configurations for open surgery, semi-open surgery, laparascopic, single port laparascopic, endoscopic and natural orifice, percutaneous and/or non-invasive (e.g., not violating body). Disclosed arms are configured to interface and control various devices, instruments and tools, and through, in part, overall known geometries, trajectories, orientations/poses, tool/base coordinates, dimensions, motions, motion patterns and pathways, and robotic arm encoder and controls data, may be further configured to calculate, register, coordinate and control and monitor/watch dog said arms. In some embodiments, robotic arms and system architectures that are configured for the histotripsy approach, allow set up in a c-arm, fluoroscopy, augmented fluoroscopy and/or cone beam CT environment as such that x-ray data acquisition may be collected during the robotic delivery of histotripsy (and avoiding collisions).

### System End-Effectors, Instrumentation and Tools

In some examples of system instrumentation and tools, utilized by one or more of the disclosed robotic arms, instruments/tools may include access devices, scissors, graspers, clip appliers, staplers, endostaplers, energy-based devices including radiofrequency, ultrasonic, microwave, with and without cutting/transection and/or cautery features, spacers, hemostats, sealants/adhesives, other various electrosurgical and ablation devices, needles, needle drivers, flexible catheter or endoscope based devices, navigation/localization devices for guiding rigid or flexible instruments, sensing devices, biopsy devices, or any other tools required for procedures. Instruments and tools may interface to the robotic arms through a variety of interface and instrument insertion and drive mechanisms and supporting architectures.

The instrument driver (e.g., instrument drive mechanism or instrument device manipulator) may incorporate electro-mechanical means for actuating the medical instrument/device and a removable/detachable medical instrument which may be devoid of any electro-mechanical components, such as motors, to allow instruments to be sterilized, but separate from the system. The driver may comprise one or more drive units arranged in axes to provide controlled torque to instruments via drive shafts (with each drive unit comprising an individual drive shaft) for interacting with the instrument, gear heads, motor, encoders to provide feedback to control circuitry, and control circuitry for receiving control signals and actuating the drive unit. Instruments may be paired to the driver using drive inputs/outputs to allow coupling through a drive interface to allow instrument coupling. In some embodiments, instrument interfaces include those designed to electromechanically interface therapy transducers, of various configuration and design (e.g., non-invasive/external body contouring, open, laparascopic, single port laparascopic, endoscopic), and where said therapy transducers may include electromechanically coupled imaging transducers, of which may also be encoded to support probe rotation, as an example.

Instruments may also be electronically keyed/coded for auto-recognition by the system, and the system software may guide, recommend and/or recognize various combinations of tools for given procedures, and conversely prompt, notify and/or warn users if the appropriate combination for a given select procedure is not selected. In some embodiments, instruments and tools may be configured to be passed/exchanged through one another (e.g., needle-based devices through a flexible endoscope actuated by the robotic arm).

Instruments may further include, but not limited to, any diagnostic, interventional or surgical tool, rigid or flexible, and any additional ancillary devices/implants to enable procedures or treatments (e.g., fiducial markers, surgical probes, tissue/cellular dyes, stains, labels, molecular probes, and/or photonic devices, etc.).

### Imaging and Visualization for Multi-Approach Systems

In some examples of system visualization and imaging devices, systems may be configured to include and/or work with various modalities and frameworks, including as examples, optical vision systems and optical flow methods, fluorescence, near-infrared, light scattering, elastic scattering spectroscopy, optical coherence tomography, endoscopic confocal microscopy, and other various biophotonic and optical modalities, raman spectroscopy, etc. Systems may also be configured to comprise, communicate or integrate with ultrasound, x-ray based systems, computed tomography (CT), cone beam CT, augmented fluoroscopy, fluoroscopy, magnetic resonance imaging (MRI), photoacoustic imaging, low frequency ultrasound/near-infrared imaging platforms (e.g., analogous to Open Water methodologies and systems), and various combinations of, including specialized image registration, fusion, flow, virtual and augmented realities of, and based on, but not limited to, various segmentation, reconstruction and image processing methods, to afford the ability to visualize the patient, procedure approach, device/treatment trajectory, the anatomical site/location surrounding/intervening and including treatment location(s), surrounding/including critical structures, the targeted disease/injury/unwanted tissue, the dynamic real-time treatment effect(s), and pre/peri/post-procedure treatment verification, and all in context to position/pose of one or more robotic arms, and through one or more user interfaces, with one or more views. In some embodiments, said visualization and positional data, as monitored by robotic encoders, may allow for automated image registration at the beginning of the procedure, or conversely, may afford the ability to return to a previous known position/pose, as time stamped earlier in the procedure, as needed/desired, and/or in an emergent situation.

### Histotripsy Therapy Systems Enabling Multi-Approach

Multi-approach robotic histotripsy therapy systems, and the core histotripsy sub-system(s), of which are configured to create, sense, enhance, modify, deliver, dynamically modulate and control histotripsy, may comprise and may configured to be based on all known methods of such, further including shock scattering, intrinsic threshold and any method of using single, multi and/or partial cycle histotripsy pulses. As well, histotripsy treatments may be directed and used with intent to partially, or fully, destroy tissue, using a minimum of one bubble cloud, of one shape/size, at one treatment location, for a specific minimum number of pulses, which may enable example applications from opening up passage ways, to removing plaques, inducing immune responses and pathways, marking tissues (e.g., as a fiducial), clearing entangled structures (e.g., tumor entangled on bile duct or vessel), treating tumors, nerves or nerve centers, treating fine or delicate structures of the eye, and any other treatment where a well-controlled tissue histotripsy effect offers clinical utility. Histotripsy treatments may also be designed to destroy specific tissues types while preserving others an ability made possible by the different energy requirements of different tissues determined by their water content, viscoelasticity and tight bonding to name some important factors.

Histotripsy therapy transducers may be configured as small form factors on endoscopic devices, rigid, semi-flexible or flexible, and on percutaneous devices or in some embodiments, may comprise larger form factors for a laparascopic surgical approach (<15 mm device), including wristed and articulating devices, an open surgical approach (e.g., <5 cm enabled on a shaft or wand), or in other embodiments may be larger (˜20 cm or greater) body contouring configurations, designed to deliver histotripsy pulses deep into the body (abdominal cavity or brain). They may comprise various geometries and shapes, as well as number of individual/discrete elements, supported by drive hardware equipped to support fixed focus and/or electronic focal steering, in one or more direction or axis. Transducers may be linear, convex or concave. Said histotripsy sub-systems may also be enabled to send and/or send/receive, including various systems/methods for cavitation mapping, and with the related drive hardware integrated into any robotic system or sub-system approach (e.g., patient side cart/robot versus vision system ancillary cart housing other core sub-systems, such as optical visualization, electrosurgical equipment, etc.).

### EXAMPLES OF MULTI-APPROACH ROBOTIC HISTOTRIPSY

### Example 1

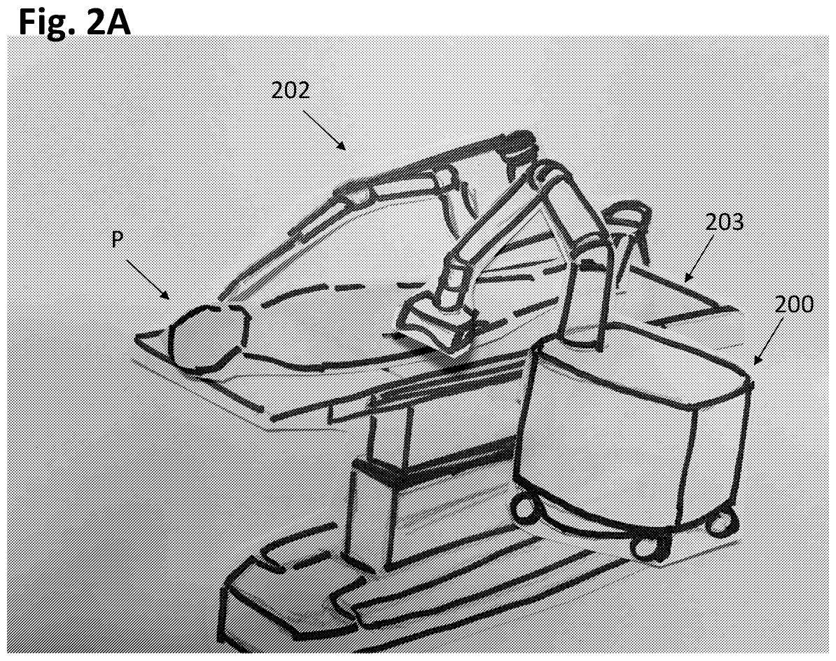

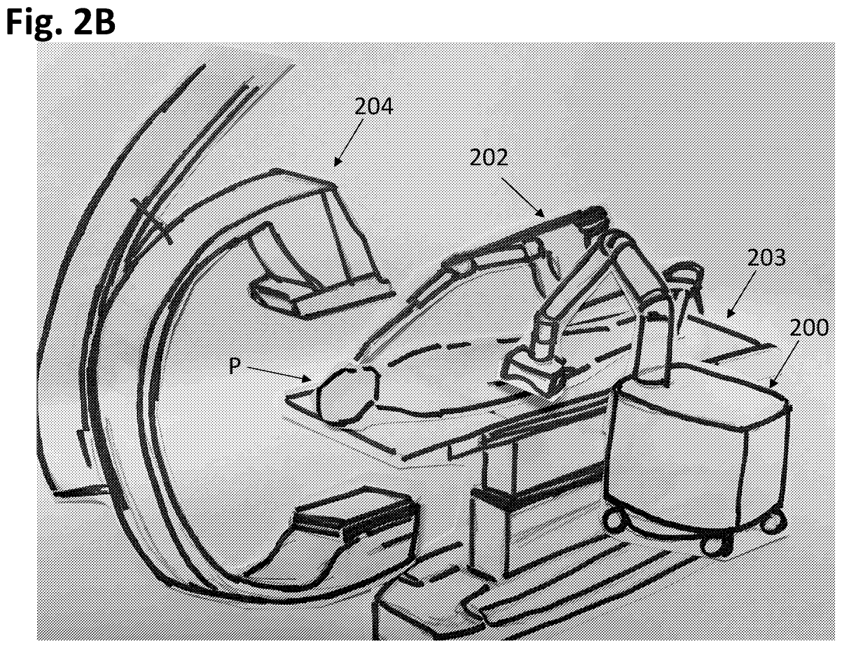

Referring to FIGS. 2A-2B, one example of multi-approach robotic histotripsy systems and procedures is provided, including a surgical system comprising a histotripsy system 200 (corresponding to the histotripsy system 100 described above) and an endoscopic (bronchoscopic) robotic system 202 configured to prepare the treatment location/site of a patient P resting on a surgical table 203.

The histotripsy system can be configured for enabling lung-directed, or any hollow/lumenal organ therapy (such as the colon) through the prepared treatment location/site. The endoscopic robotic system, using navigation and positional/localization sensing capabilities, allows a user such as a physician or surgeon to access any desired hollow organ location. For example, in a lung treatment, the endoscopic robotic system can access any airway location (and level) in the lung, of which comprises one (or more) targeted suspicious lung nodules (or known cancers), and at the lobar, segment or sub-segmental level, allowing the user to fluidize the airway(s) to create an acoustic window within that selected level/anatomical location. These specific procedure steps are aimed at preparing the location to adequately receive acoustic therapy, including histotripsy. Fluidizing the airway(s) may comprise using an biocompatible medium, including saline, buffered saline and/or other aqueous mediums, and mediums will also be configured to be of acceptable oxygen/gas saturation, and may be degassed to do such. The method of fluidizing may include navigating to a predetermined location/bi-furcation of the airways, and may optionally include mechanically blocking/sealing proximal locations from fluid. The fluidizing and/or blocking location may be referenced on a registered image (e.g. CT scan or optical image), and may be tracked/monitored in real-time through navigation, direct optical visualization and/or through a fluoro or cone beam CT (using X-ray monitoring).

The endoscopic robotic system, in some configurations, may allow the fluidization steps to be executed with an imaging system 204 under continuous real-time visualization (e.g., optical camera, catheter-based ultrasound, etc.) and localization of position in context to a segmented airway tree (e.g., electromagnetic navigation, shape sensing, etc.), as well as under full field of view using augmented fluoroscopy and cone beam CT (e.g., visualize entire thoracic cavity, any CT to body divergence, etc.). In some configurations, the visualization features may need to be removed prior to additional instruments being inserted. Additional devices, used through the working channel of the bronchoscopic robot or endoscope, inserted and exchanged one or more times, may be also used to conduct these steps. This may further include balloon catheters or other devices enabled to deliver acoustic coupling medium, as an example, degassed water or saline, as well as to allow for sealing the fluidized lung compartment (at lobe, segment or sub-segment level) with fluid.

Working in concert with the endoscopic/bronchoscopic robotic system, the non-invasive histotripsy robot can be positioned over the target hollow organ location (e.g., over the chest wall for lung treatments), with its pose and position aligning the geometric focus of the histotripsy therapy transducer to the user selected and defined hollow organ target. The histotripsy robot can then be enabled to deliver histotripsy pulses and treatment through the acoustic window afforded via the endoscopic robotic system. In some embodiments and methods, some or all of these steps are executed with the patient positioned on a bed allowing augmented fluoroscopy and cone beam CT data to be acquired, and where data is acquired while one or both the robotic approaches are in place. The augmented fluoroscopy and cone beam CT may be used to assist in planning, treatment and treatment verification.

### Example 2

In another embodiment similar to Example 1, the bronchoscopic robot comprises the Monarch robot (Auris Health, JNJ). This specific example provides concurrent continuous optical imaging, electromagnetic navigation and working channel access for all fluidics required procedure steps. In this example, the use of radial probe endobronchial ultrasound may be used to visualize the anatomical site, treatment effect (e.g., histotripsy bubble cloud) and tissue effect (change in tissue reflection/scatter following treatment).

### Example 3

In another embodiment similar to Example 1, the bronchoscopic robot comprise the Ion robot, (Intuitive Surgical). This specific example provides concurrent optical imaging and shape sensing localization, but further requires the camera system to be removed to allow any fluidics delivery devices to be inserted into the working channel, for any fluidics support devices and related steps (e.g., using a balloon catheter to deliver degassed water and seal the proximal airway). In another related example, this multi-approach may be configured to use an endoscopic robot (Ion) and a histotripsy bed side cart robot (non-invasive transcostal approach), all in a cone beam CT environment, where the cone beam CT is used to acquire images for planning, lung preparation and fluidization, navigation, device localization, and visualization of treatment pre, peri and post histotripsy. The cone beam may also be used to calculate pose/position of the transducer and predict treatment locations based on such, as well as register ultrasound data with the cone beam data (and synchronized with the robotic arm positional encoders).

### Example 4

In additional examples similar to Examples 1-3, multi-approach systems for lung treatment and including the use of exemplary robot systems such as Monarch (Auris Health, JNJ) or Ion (Intuitive Surgical), the histotripsy robot may be further configured with multi-aperture ultrasound imaging (MAUI), to allow users to visualize the targeted and surrounding lung tissue. The MAUI imaging feature may be configured to be a MAUI imaging probe, mounted co-axial to the histotripsy therapy transducer, both mounted on the distal end of the histotripsy system robotic arm.

### Example 5

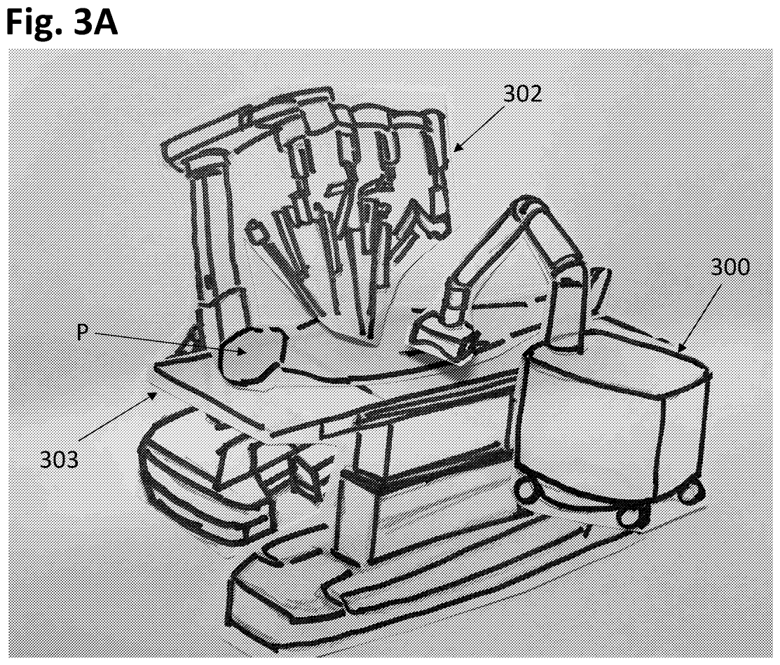

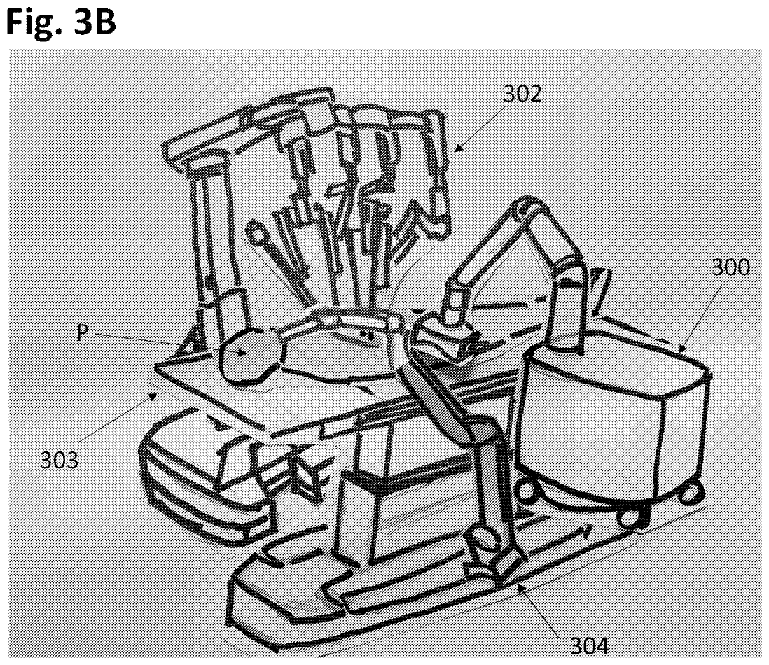

In another related embodiment to Examples 1-4, referring to FIG. 3A, a surgical system can include a histotripsy system 300 (corresponding to the histotripsy system 100 described above) and a laparascopic robot 302 configured to access and prepare any hollow organ (such as a lung) of a patient P for treatment. As described above, the patient can rest on a surgical table 303. In the embodiment of lung treatment, the laparascopic robot can prepare the lung for a wedge, sub-segmental, segmental, or lobar level resection using a master/slave configuration (e.g., Da Vinci Xi, Intuitive Surgical). In combination, the histotripsy system (patient side cart with user console), can be configured to treat the hollow organ (e.g., related lung tumors and nodal stations) immediately prior to laparascopic surgery. The Da Vinci robot can be further used to allow acoustic coupling for the histotripsy treatment, via fluidizing the body cavity of the patient adjacent to the target hollow organ (e.g., the thoracic cavity prior to histotripsy application in the lungs). As described above, the surgical system can include an imaging system to allow for visualization during treatment. In some embodiments, endobronchial ultrasound (EBUS) is used to observe nodal stations/tumors pre/peri/post-histotripsy.

### Example 6

Similar to Example 5, another embodiment is provided in FIG. 3B in which the surgical system can comprise a histotripsy system 300, a laparascopic robot 302 (and arm) configured to access and operate on a target hollow organ such as the lung, and further including a robotic endoscopic system 304 (flexible). The robotic endoscopic system can be configured to fluidize the hollow organ itself. In this embodiment, if the target hollow organ is the lung, the robotic endoscopic system can be configured to fluidize the lung, which may include the lung itself (whole lung, lobe or sub-lobar), and/or the thoracic cavity (extra-lung), to allow enhanced acoustic coupling for treatment directed in/through the lung. The example of FIG. 3B, the laparascopic robot 302 can be configured to access the target hollow organ laparsacopically, and further includes the ability to fluidize the patient cavity surrounding or adjacent to the target organ (e.g., the thoracic cavity in the event of lung treatment). Similarly, the endoscopic robot allows for access within the target hollow organ itself, including the ability to fluidize the hollow organ. The histotripsy treatment may be delivered via one of the plurality of additional robotic arms, or a separate bed side cart. In another related example, the surgical system may include any of the imaging systems described above, including ultrasound, CT, fluoroscopy, a multi-aperture ultrasound imaging, etc.

### Example 7

In another example comprising a configuration in the spirit of Example 6, includes the system(s) and methods for delivering liver-directed histotripsy treatments through significant transcostal acoustic blockage. This example may further include scenarios where the histotripsy therapy transducer may be positioned with full rib coverage (e.g., maximum acoustic blockage from ribs), and the airway(s) and/or thoracic cavity fluidized to further provide a better acoustic window into and through the lung, and of which may enhance abdominal directed treatments which may share a similar pathway. In another related example, the robotic arm enabled with the histotripsy therapy transducer may comprise a multi-aperture ultrasound imaging probe configured to visualize into the lung parenchyma and into the liver.

### Example 8

In another example, a multi-arm laparascopic robot of a bed, column or cart based architecture, including one arm coupled with an endoscope for visualization, is configured to observe the disappearance of a molecular/surgical probe, during and/or after treatment via non-invasive (extracorporeal) histotripsy, enabled from a second robotic arm. In one embodiment, the surgical probe is a near-infrared probe which allows for direct fluorescent visualization of the labeled tissue/cells (e.g., specifically labeled tumor cells). In another embodiment, the overall system configuration (laparascopic and non-invasive) affords the ability to visualize the probe and the surgical end-effector and tissue, and the echogenicity, as well as any changes in echogenicity of the targeted tissue or bubble cloud under B-mode ultrasound (from the non- invasive arm), concurrent to the near-infrared probe through the vision system of the laparascopic arm, and as tumor/tissue is destroyed, the appearance of the molecular/surgical probe (disappearing), may offer immediate treatment verification of tissue effect.

### Example 9

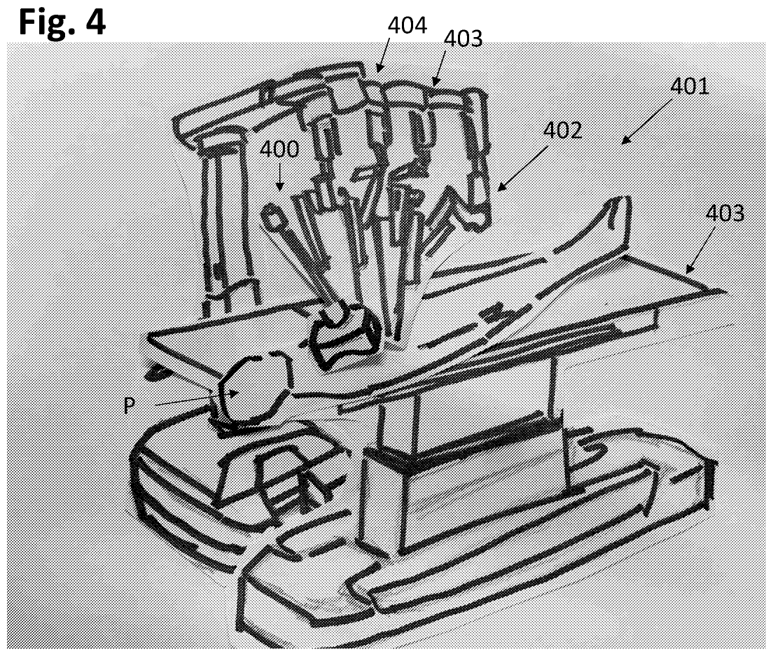

In another example, referring to FIG. 4, a robotic system 401 can include many of the robotic features/functions described above. In one embodiment, the robotic system can include 1) one or more robotic arms for 402 configured for a laparascopic approach, 2) one or more robotic arms 403 configured for an endoscopic approach, 3) one or more robotic arms 400 configured for a non-invasive (histotripsy) approach, and 4) one or more arms 404, manual or robotic, for patient access/coupling, including acoustic access/coupling. In contrast to the systems described above in which the endoscopic/laparascopic/histotripsy robots are separate, this embodiment comprises a single robotic system with a plurality of arms for each sub-system.

The robotic system 401 may be set up in a variety of orientations and perspectives, allowing multi-perspective procedures with minimized arm collisions and enhanced setup and ease of use. The respective robotic arms may be controlled from a single master, in a master/slave configuration, or different arms (and associated robotically-enabled tools/end-effectors) may be actuated/controlled through more than one user interface or console. In some embodiments, the robotic system can comprise a bed based robot system. In other embodiments, the robotic system can be configured as a patient side based robot (e.g., Da Vinci, Intuitive Surgical). The robotic system can be configured with surgical instruments, a visualization/imaging probe (e.g., optical, ultrasound) and a histotripsy therapy transducer, and the primary user interface and controls system is the robot master.

### Example 10

In another example similar to Example 8, but in a configuration where each specific tool is coupled, actuated and controlled through a dedicated bed side cart, with all carts tele-remotely connected to one or more user input devices or masters. In some embodiments, the histotripsy system may be controlled through a dedicated user interface/console. In other embodiments, all robotic arms and devices, are controlled through a single master.

### Example 11

One or more of the potential configurations disclosed herein, are used to treat pancreatic tumors, and configured to deliver histotripsy to convert early or mid-stage medically inoperable patients to an operable state, via allowing better surgical access and treatment to tumor related blood vessels, including skeletonizing vascular involved tumor, without damaging vascular structures, pancreatic ducts, biliary system, or sensitive bile ducts. By doing such, tumors which typically would have rendered too much clinical patient risk and injury (due to tissue bleeding perforations, unintended collateral injury, may be rendered operable by allowing better preparation and management of critical structures to minimize potential adverse events.

For example, in one embodiment, a multi-approach robotic system including laparascopic/endoscopic/histotripsy systems can be configured to convert a medically inoperable patient to an operable patient. There may be many reasons for the patient being medically inoperable, which can include inability to access tumor related blood vessels or sensitive intra-organ lumens or ducts. In some examples, the target organ (e.g., a pancreas) can be visualized either internally or externally with an endoscopic robotic system, as described above. Next, histotripsy therapy can be applied to target regions of the target organ to liquefy or lyse the soft tissues. The histotripsy can be specifically tailored to target only the soft tissues and not the blood vessels, ducts, lumens, etc. For example, by controlling the histotripsy pulses to attain cavitation above only a specific threshold, only the soft target tissues can be dissolved, leaving the vessels, ducts, etc. undisturbed. Upon completion of the histotripsy therapy, a laparascopic robotic system can be used to operate on the remaining tissue structures (e.g., blood vessels that feed a target tumor, sensitive ducts, lumens, etc.).

### Example 12

One or more of the multi-approach histotripsy robots, wherein similar to Example 11, the multi-approach robot configurations allow de-bulking of pancreatic tumors and stroma to afford increased tumor perfusion and enhanced drug delivery. In this example, histotripsy may be used to destroy the soft tissue and cellular component of the tumor(s) and surrounding matrix components, to reduce interstitial and intratumoral pressure. For more mechanically resilient matrix structures and architectures, including tumor and adjacent tumor tissue, varied pulse sequences may be used to exert desired damage/tissue effect. Further, the specific bubble cloud pattern and pathway (moving the bubble cloud through the pattern) may be modified to deliver specific spatial patterns, including partial treatments (and/or varied dose within them, e.g., number of total pulses), to control extent of tissue effect. Alternatively, an ablation cavity can be created for the local instillation of chemo and or immunotherapeutic agents.

### Example 13

A multi-approach robot configured to allow surgeons to visualize the pancreas and liver directly, including the use of an endoscope, while concurrently treating with histotripsy, non-invasively. In some procedures, histotripsy treatment may be configured to prepare the target organ systems for resection, where preparation may include skeletonizing the organ to better enable resection (e.g., enhanced blood vessel and bile duct management) and minimize potential adverse events (e.g., bleeding or bile leak). In other procedures, histotripsy treatment may be used to divide tissue much like a scissors or scalpel leaving vessels intact for later treatment with commonly employed ligation devices such as sutures, clips, energy based ligation devices such as Bipolar, monopolar, ultrasonic and microwave) and stapling devices.

### Example 14

A multi-approach robot configured with a plurality of robotic arms to perform tissue sparing surgery of the kidney, where two or more laparascopic robot arms are configured and coupled to laparascopic tools/instruments, including visualization, and one or more robotic arms, configured for non-invasive histotripsy. In some embodiments, the multi-approach robot may be configured with flexible endoscopy-enabled robotic arms and drive systems, to visualize the inside of the kidney, pre, peri or post-histotripsy. In some embodiments, the robotic system may use image guidance, including, but not limited to ultrasound and ultrasound fusion with CT and/or MRI, with the real-time ultrasound imaging registered to the positional data obtained the robotic arm encoders.

In another example, for methods of using combined laparascopic and non-invasive approaches, but from a single system, a robot may be equipped for a minimum of a three arm approach, with one arm for laparascopic visualization of the kidney and work space, one or more laparascopic surgical tools and one histotripsy transducer. In this embodiment, all devices may be controlled through a single master/console.

### Example 15

An example, including any of the above examples or multi-approach robotic system(s) configurations that may be envisioned, wherein the robotic system, or one of the multi-robotic form factor systems, using a freehand ultrasound component to help plan or direct therapy. And in some embodiments, where said freehand ultrasound device(s) are tracked positionally, in some cases in 6 or more degrees of freedom, and further registered to other ultrasound images or video, or other imaging modalities (e.g., optical, CT, MRI, etc.).

### Example 16

A multi-approach system is configured to afford overcoming today's challenges of treating hemorrhage or clot in the brain, and wherein a catheter based, bed/table based (integral to table side and/or next to table) robotic drive system, in combination with a non-invasive transcranial histotripsy system approach (bed/table side), are used to liquefy and aspirate hemorrhage, clot or thrombus. In another related example, this multi-approach configuration may be conducted within a cone beam CT, and as one integrated system approach.

### Example 17

In some embodiments of Example 16, the catheter based robot is a Corindus/Siemens robot. In other embodiments, it may be a Hansen endovascular/neurovascular enabled robotic system. Further, in some cases, one or more of the robots (catheter or histotripsy) may be commanded tele-remotely, ranging from the local control room (controlling also the cone beam CT) and/or from a distance (e.g., another care center).

### Example 18

The examples of a combination of neuro-endo robotic approach and non-invasive transcranial histotripsy, wherein the application is to treat and remove/aspirate tumor remnant or lysate from the brain, to relieve pressure, open anatomical structures (e.g., ventricles) and/or remove aggressive disease pathologies from the affected/surrounding sites. Conversely, the following such, the neuro-endo robot may be used to deliver therapy and return to previous histotripsy treatment sites, including medical, immunotherapy, cell therapy, local radiation and/or any combinations of.

### Example 19

Examples similar to Examples 16-19, but wherein the treatment locations are sub-dural or epidural and very shallow such as meningioma, subdural hematoma and epidural hematoma.

### Example 20

Further examples wherein the catheter based robot provides navigation and access for delivery of a catheter hydrophone, to facilitate localization of the bubble cloud, minimally invasively, and to enable a robotic arm configured with a histotripsy transducer. This multi-approach system may be further configured as one overall system or as collaborative robots/approaches working in concert.

### Example 21

A surgical robot configured with a minimum of 4 robotic arms, with an arm coupled to a visualization device, tissue manipulator (e.g., grasper), clip applier and/or vessel sealer and a histotripsy transducer, together enabled for an abdominal approach and resection of visceral tissue. In one specific example, the histotripsy transducer is used to provide instantaneous tissue cavitation and skeletonization, as the clip applier/vessel sealer follows closely behind sealing the skeletonized tissue (e.g., liver).

### Example 22

A general example, of where a laparascopic robot (patient side cart) may be used to stabilize and hold organs/tissues within a fluid filled endo-bag or containment device (laparascopically), and wherein a second histotripsy therapy transducer, applied non-invasively, may be used to liquefy/destroy the tissue contained within the endo-bag or containment device. Alternatively, the second histotripsy therapy transducer may be applied directly to the fluid filled bag or containment device.

### Example 23

An example, wherein a multi-approach robotic approach for thyroidectomy is conducted, wherein, a percutaneous approach with surgical instrumentation is used to remove complex/mixed morphology tissue, and a non-invasive histotripsy transducer is actuated with a robotic arm enabled to couple/manipulate/direct said histotripsy transducer above the thyroid, external to the body. Complex/mixed morphology tissue may include hetereogenous tissue as observed on ultrasound, and where histotripsy is used to direct tissue treatment within desired/user defined zones within the hetereogenous region, and then aspirated/removed through percutaneous approach. The user may continue/repeat treatment, as desired, based on the real-time feedback in tissue changes.

### Example 24

An embodiment, wherein a laparascopic robot is configured with surgical instrumentation for resection of the prostate, and one arm of the robot comprises a non-invasive histotripsy transducer for trans-perineal treatment. In a related but alternative embodiment, a transrectal histotripsy transducer is utilized for the same procedure, and to enable laparascopic resection of the boundaries and margin, and aspiration of any undesired residual tissue remnants.

Additional unlimited examples for systems and methods of multi-approach histotripsy robotic systems can be envisioned, across the body, and approach, and these examples are not intended to be limiting.

### Example 25

An embodiment, wherein a laparascopic robot is configured with surgical instrumentation for resection of colorectal tumors, and one arm of the robot comprises a non-invasive histotripsy transducer for trans-perineal treatment. In some embodiments, the colon can be fluidized prior to the histotripsy treatment to provide an acoustic window into the colon. In a related but alternative embodiment, a transrectal histotripsy transducer is utilized for the same procedure, and to enable laparascopic resection of the boundaries and margin, and aspiration of any undesired residual tissue remnants.

What is claimed is:

1. A method of treating tissue of a patient with a robotic surgical system, comprising the steps of:
identifying a target tissue location with an imaging sub-system of the robotic surgical system;
preparing the target tissue location for histotripsy therapy with a laparascopic sub-system of the robotic surgical system; and
delivering histotripsy therapy to the prepared target tissue location with a histotripsy sub-system of the robotic surgical system.

2. The method of claim 1, wherein the imaging sub-system comprises an endoscopic robotic system.

3. The method of claim 1, wherein the imaging sub-system comprises an ultrasound imaging system.

4. The method of claim 1, wherein the imaging sub-system comprises a CT imaging system.

5. The method of claim 1, wherein the imaging sub-system comprises an augmented or enriched multi-modality imaging system.

6. The method of claim 1, wherein the imaging sub-system comprises a fluoroscopy imaging system.

7. The method of claim 1, wherein the imaging sub-system comprises an imaging device disposed on a robotic arm of the robotic surgical system.

8. The method of claim 1, wherein preparing the target tissue location further comprises resecting intervening tissues between an exterior of the patient and the target tissue location.

9. The method of claim 1, wherein the target tissue location comprises a hollow/lumenal body organ, vessel, or lumen, and wherein preparing the target tissue location further comprises fluidizing the target tissue location with the laparascopic sub-system to create an acoustic window within the target tissue location and/or pathway to the location.

10. The method of claim 1, wherein delivering histotripsy therapy further comprises lysing or liquefying the target tissue location.

11. The method of claim 1, wherein the target tissue location comprises a first tissue structure and a second tissue structure, wherein delivering histotripsy therapy further comprises lysing or liquefying the first tissue structure but not the second tissue structure.

12. The method of claim 11, wherein the first tissue structure comprises soft tissue.

13. The method of claim 11, wherein the first tissue structure comprises cancerous tissue.

14. The method of claim 11, wherein the first tissue structure comprises tumor tissue.

15. The method of claim 11, wherein the second tissue structure comprises blood vessels.

16. The method of claim 11, wherein the second tissue structure comprises ducts including bile ducts.

17. The method of claim 1, wherein delivering histotripsy further comprises:
evaluating a cavitation threshold at one or more locations within the target tissue location; and
optimizing histotripsy therapy parameters based on the evaluated cavitation threshold.

18. The method of claim 1, wherein the histotripsy sub-system is disposed on a robotic arm comprising 3 or more degrees of freedom.

19. The method of claim 1, wherein the robotic surgical system comprises a cart/column based surgical system.

20. The method of claim 1, wherein the robotic surgical system comprises a bed-based surgical system.

21-70. (canceled)
